(19) United States
(12) Patent Application Publication
(10) Pub. No.: US 2019/0321291 A1
(43) Pub. Date: Oct. 24, 2019

(54) 3D Printing and Drug Delivery

(71) Applicant: Biogelx Limited, Lanarkshire (GB)

(72) Inventors: Michael Leo Connolly, Lanarkshire (GB); Laura Goldie, Lanarkshire (GB); Mhairi Malone Harper, Lanarkshire (GB); Eleanore Jane Irvine, Lanarkshire (GB); David Lightbody, Lanarkshire (GB)

(73) Assignee: Biogelx Limited, Lanarkshire (GB)

(21) Appl. No.: 16/467,620

(22) PCT Filed: Dec. 8, 2017
(86) PCT No.: PCT/EP2017/082101

§ 371 Date: Jun. 7, 2019

(30) Foreign Application Priority Data
Dec. 9, 2016 (GB) 1620979.3
Oct. 13, 2017 (GB) 1716852.7

(51) Int. Cl. A61K 9/06; A61K 47/18; A61L 27/52; A61M 5/19

(52) U.S. Cl. CPC A61K 9/06; A61M 5/19; A61L 27/52; A61K 47/183

(57) ABSTRACT

A 3D structure of a hydrogel for supporting cell growth or for use in sustained drug delivery is formed using peptides and/or peptide derivatives that self-assemble via cross-linking into a stiff gel. The hydrogel structure is formed using a method based on 3D printing. A hydrogel precursor is extruded under conditions to generate a hydrogel, by extruding a solution of the peptides into a solution containing cations, whereby the cations enable cross-linking of pi-stacked peptides, or by co-extruding it with the cations, the peptides and cations being mixed only at the point of co-extrusion. The stiffness of the hydrogel can be tuned by adjusting the combination of peptides, either by the selection of peptides or combinations thereof, or the proportions of the combinations, and/or by adjusting the proportion of cations present.

This application is a U.S. national stage application filed pursuant to 35 U.S.C. § 371 from International Patent Application PCT/EP2017/082101, filed on Dec. 8, 2017 which claims the benefit of priority and the filing date of United Kingdom Patent Application GB 1716852.7, filed on Oct. 13, 2017, and United Kingdom Patent Application GB 1620979.3, filed on Dec. 9, 2016, the content of each of which is hereby incorporated by reference in its entirety.

### INTRODUCTION

The present invention relates to creating three-dimensional (3D) structures by printing processes and to drug delivery using such structures. In particular, the invention relates to printing cell growth structures that contain cells and/or active agents and to injectable structures for in vivo drug delivery.

### BACKGROUND TO THE INVENTION

3D bioprinting utilises 3D printing technology to produce functional miniaturised tissue constructs from biocompatible materials, cells and supporting components such as cell media. Major applications include use in high-throughput in vitro tissues models for research, drug discovery and toxicology, in addition to regenerative medicine/tissue engineering applications. It involves layer-by-layer, precise positioning of the biomaterials and living cells, with spatial control of the placement of functional components. The technology has made significant progress towards the clinical restoration of tissues and even organs such as ear, nose, bone, and skin.

A vital yet limiting component in effective 3D printing of cells, and ultimately miniaturised tissues, is a scaffold material (essentially ‘printer ink’) to form the 3D structure to support the cells. Such a material must offer properties suited to both the 3D printer mechanism and to hosting or maintaining the viability of the cells within the scaffold structure. Obtaining such a balance of properties is the main challenge within 3D bioprinting, and is reliant on a number of key features.

There are two classes of materials commonly used for 3D bioprinting. The first class is curable polymers, which can be extruded by a thermal process and are often used for scaffolding purposes. Cells are seeded after printing on these scaffolds so that cells grow within to generate tissue constructs or used as is for implantation.

The second class comprises materials that store a large amount of water (up to >99%) and provide a favourable environment for the cells. Hydrogels belong to this class, and are used to encapsulate living cells. Cell-laden hydrogels are typically referred to as a “bio-ink” and hydrogels that solidify through thermal processes, photo-cross-linking, or ionic/chemical cross-linking may be used to make bio-inks.

These hydrogel inks can be based on either natural polymers (including alginate, gelatin, collagen, and hyaluronic acid often isolated from animal tissues) or synthetic molecules (e.g. PEG). Whilst the obvious advantage to employing naturally-derived hydrogels is their inherent bioactivity, the resulting printed constructs have often shown a lack of mechanical integrity, and the ability to tune the properties of such hydrogels is limited. In addition to this, animal-derived hydrogels often have issues with batch-to-batch reproducibility.

Alginate gels are used in 3D printing, especially in extrusion-based printing. Historically, however, the printed cells in the hydrogels failed to degrade the surrounding alginate gel matrix, causing them to remain in a poorly proliferating and non-differentiating state.

Some researchers have utilised 2-component hydrogel mixtures in an attempt to achieve a good balance of mechanical properties and cell compatibility (e.g. PEG and hyaluronic acid mixtures). However, a single component bio-ink that is non-animal derived, possesses suitable mechanical and gelation properties, and appropriate physical/chemical features for compatibility with a range of cell types has yet to be realised.

Again, while discussing 3D structures for cell growth, there appears to be no indication that these mixtures can be used in 3D printing techniques.

CN105238132 describes biological ink for 3D printing. The biological ink comprises water-soluble synthetic polymers for cross-linking water-soluble natural polymers. The biological ink is said to overcome the defects that traditional 3D printing ink is single in component structure, does not have good biological activity and needs to utilize organic solvents. The hydrogel obtained through curing of the ink (by photo-mediated cross-linking) is said to have controllable mechanical properties and good structural stability.

It is separately known to support cell growth on structures which resemble physiological conditions, such structures including gels. Zhang et al (J. Am. Chem. Soc. 2003, 125, pp 13680-13681) describes protected dipeptides, for example Phe-Phe, Ala-Ala, Gly-Gly, Gly-Ala, Gly-Ser in combination with an aromatic stacking ligand, forming fibrous scaffolds. These were carried out at a pH of 3-5, which is too acidic for normal cell growth. No investigations were carried out at physiological pH. Other peptide-based gels are described in Gary Scott et al, Langmuir, vol. 29, 2013, pages 14321-14327; Mi Zou et al, Biomaterials, vol. 30, 2009, pages 2523-2530; and Apurba Das et al, Faraday discussions, vol. 143, 209, page 293-303. Zhou et al (J. Tissue Eng., Vol. 5, 2014) discusses hydrogels based on combinations of Fmoc-FF and Fmoc-RGD.

U.S. Pat. No. 8,420,605 in the name of the University of Strathclyde describes further hydrogels which comprise an aromatic stacking ligand, such as Fmoc or Cbz, together with peptides or peptide derivatives at physiological pH. A hydrogel was formed at pH 7 from Fmoc-Phe-Phe. Further work was carried out using other peptides, in combination with an aromatic stacking ligand, such as C-Phe-Phe, where C=Alanine, Valine, Leucine or Phenylalanine. The hydrogel formed from Fmoc-Phe-Phe was, however, too hydrophobic for attachment of cells and thus did not form a suitable scaffold for cell growth of anchorage dependant cells.

WO 2013/124620 describes making a hydrogel of pH greater than 8, liquefying the hydrogel so cells can be added and then reforming the gel.

WO 2013/072686 is directed to 6-10 amino acid-containing peptide-based hydrogels of pH 2-3 in which the peptides coalesce such that they ‘self-assemble’ to form a hydrogel. Such hydrogels are said to be used to deliver pharmaceutically active compounds through mucosal tissue.

“Controlled patterning of peptide nanotubes and nanospheres using inkjet printing technology”, Adler-Abramovich, L.et al., J. Peptide Sci., Vol. 14, 2008, p 217-223 describes the printing of aromatic dipeptide nanotubes, including F-F, Fmoc-FF and Boc-FF using an inkjet printer. Solutions of the dipeptides were made in HFP and 50% ethanol and following printing nanotubes and nanospheres were achieved, upon evaporation of the solvent. The materials printed were not in the form of hydrogels.

“Self-assembled peptide-based hydrogels as scaffolds for proliferation and multi-differentiation of mesenchymal stem-cells”, Yung-Li Wang et al., Macromol. Biosci., Vol. 17, 2017, article 1600192, describes the culture of mesenchymal stem cells in mixtures of Fmoc-FF and Fmoc-RGD hydrogels. This papers does not suggest the use of such materials in 3D printing.

“The effect of calcium chloride concentration on alginate/Fmoc-diphenylalanine hydrogel networks”, Celik, E. et al., Materials Sci. Eng. C, Vol. 66, 2016, p 221-229 describes the release of Vancomycin from Fmoc-FF/alginate gels, and how the release rate is influenced by the extent of crosslinking, which is controlled by CaCl₂ concentration. The paper does not suggest that such materials are suitable for 3D printing. “Calcium ion triggered co-assembly of peptide and polysaccharide into a hybrid hydrogel for drug delivery”, Yanyah Xie, et al., Nanoscale Res. Letts., Vol. 11, 2016, p 184, also relates to hydrogels comprising a combination of Fmoc-FF and alginate and again discusses the use of calcium ions to cross link both the peptides and the alginate. Again there is no suggestion that these materials could be used for 3D printing.

“Short to ultrashort peptide hydrogels for biomedical uses”, Wei Yang Seow, et al., Mat. Today. Vol. 17, 2014, p 381-388 is a review of peptide hydrogels in biomedical uses. This document speculates that such hydrogels have the potential to be used in bioprinting, however, there are no details of how such printing can be carried out and the requirements for the printing.

For optimising of cell growth such hydrogels must form a sufficiently stiff gel to support cell growth and must do so under physiological pH. In addition, it is advantageous for the gel to be optically transparent to aid monitoring of the cells, not be too concentrated (as low concentration of gel components or large pore sizes between gel components can be beneficial for cell growth), and have sufficient longevity for the growth required.

Sustained release drug delivery system can be a major advance toward solving the problem concerning drugs with a short half-life that are eliminated quickly from blood circulation and require frequent dosing. Sustained release formulations have been developed in an attempt to release the drug slowly and maintain a near-constant drug concentration for long periods of time.

Sustained release systems include any drug delivery system that achieves slow release of drug over an extended period of time. If the system is successful in maintaining constant drug levels in the blood or target tissue, it is considered as a controlled-release system.

Known sustained release systems are based on hydrophilic cellulose-based polymers, hydrophilic non-cellulosic polymers including alginates and gums and hydrophobic polymers.

It is the aim of the current invention to provide alternative, preferably improved, structures for support of cell growth, both in vitro and in vivo, as well as methods for making the same. A separate though related aim is to provide alternative materials for drug delivery, especially improved materials for sustained drug release.

### SUMMARY OF THE INVENTION

The present invention relates to using self-assembling peptide amphiphiles, cross-linked e.g. by cations into stiff gels, in bio-ink, cell growth and drug delivery.

The invention preferably employs such aromatic peptide amphiphiles containing a short (e.g. di- or tri-) peptide sequence, with the N-terminus capped by a synthetic aromatic moiety. In such compounds, self-assembly is based upon aromatic π stacking interactions, and the propensity of the peptides to form a β-sheet type hydrogen bonding arrangement in association with a cross-linking agent, preferably a cation.

Methods of printing use a mixture of hydrogel precursor and activator, wherein the hydrogel precursor comprises a plurality of peptide derivatives and the activator comprises a cross-linking agent.

A mixture of hydrogel precursor and activator and cells/drug is used (whether gelled or non-gelled) for cell growth and in sustained release drug delivery.

### DETAILS OF THE INVENTION

According to the invention there is provided a method of printing, comprising:
a. preparing ink comprising a mixture of hydrogel precursor and activator in non-gelled form,
b. printing the ink onto a substrate, and
c. allowing the ink to gel,
wherein the hydrogel precursor comprises a plurality of peptide derivatives and the activator comprises a cross-linking agent.

Inks of embodiments of the invention can be gelled in situ. Preparing the ink preferably comprises combining the hydrogel precursor with activator so that it begins to gel, and the printing comprises printing the gelling ink before it has fully gelled. Gelling generally takes place over seconds and minutes, allowing time for printing before the gel is too viscous.

As such the term “non-gelled” encompasses liquids and partial gels whereby some initial gelling has occurred to the extent that the ink can hold its shape, but not to the extent that printing is prevented.

Preparing the ink also preferably comprises combining the hydrogel precursor with the activator so that it partially gels, and allowing the ink to gel comprises combining the partially gelled ink with further activator (the same or different to that used in step a) so that a gel is formed.

Preferably the ink may be printed into a solution containing the same or a different activator, or may be placed in such a solution immediately or after some delay following printing. As a preferred additional or alternative step, the printed ink may be sprayed or otherwise contacted with additional activator, especially as a solution.

To combine the gel components, the method can include co-printing or coextruding hydrogel precursor and activator from separate reservoirs. In this way they are contacted with each shortly before printing and gelling.

Combinations of these steps are also envisaged whereby a hydrogel precursor and activator in non-gelled form is co-printed or co-extruded with an activator from separate reservoirs. Such printed ink may then be exposed to further activator as set out above.

Inks of embodiments of the invention can alternatively be gelled in advance and then converted into a printable form. Thus preparing the ink for printing optionally comprises fluidising or liquefying a gelled hydrogel. In this way for example the methods may comprise agitating a gelled hydrogel so as to fluidise it, and printing the fluidised ink before it has re-gelled. Suitable forms of agitating comprise subjecting the gelled hydrogel to one or more or all of (i) sonication, (ii) vibration and (iii) increase in pressure, so as to render it sufficiently fluid to be printed.

According to the invention there is similarly provided a method of printing, comprising
a. preparing ink comprising a hydrogel precursor in non-gelled form,
b. printing the ink so as to contact it with activator, and
c. allowing the ink to gel,
wherein the hydrogel precursor comprises a plurality of peptide derivatives and the activator comprises a cross-linking agent.

To achieve gelling the precursor must be combined with the cross-linking agent. In one method this is done by printing into a solution containing the activator. The printed ink may also be placed immediately, or after some delay, into a solution containing the activator. Additionally or alternatively the printed ink may be sprayed or otherwise exposed with additional activator, e.g. as a solution. Hence, the combining of the gel components takes place after extrusion/printing. In other methods this is done by preparing ink as a combination of hydrogel precursor with an amount of activator so that it partially gels, completion of gelling occurring after contact with further activator in step b.

To combine the gel components, the method can include co-printing or coextruding hydrogel precursor and activator from separate reservoirs. In this way they are contacted with each shortly before printing and gelling. The contact may occur at the point of printing, i.e. at the end of the extrusion process, or initiation of printing, i.e. at the start of the extrusion stage of printing.

The combination of these is also envisaged, such that a hydrogel precursor and an activator are co-printed, according to any method described above, and the printed ink is then subjected to further activator, as set out above.

The peptide derivatives, as used in examples, preferably comprise at least 2 amino acids or derivatives thereof linked to a third component which is an aromatic group, an aromatic amino acid or an aromatic stacking ligand. Independently, the activator used in examples comprises a cross-linking agent, e.g. a cation. Typically, the cation is Ca²⁺, Mg²⁺ or other cations typically found in cell culture media, for example Li⁺ or Na⁺. Additionally or alternatively, the cation may also be the cationic part of a zwitterion, for example in an amino acid. As a specific example used below IKVAV, a fragment of laminin, is a zwitterion, and can also be used as the activator. IKVAV is a gel forming protein and may be used to cross-link the peptide derivatives through its cation region, its positively charged group(s). Other peptides having a cation, typically as part of a zwitterion, can also be used as the activator. Combinations of different activators, for example both IKVAV and metal cations, can also be used. Combination of the peptides in the presence of the activator, e.g. cation, leads to cross-linking and formation of viscous gels.

In preferred embodiments of the invention the peptides derivatives are of formula I

ASL-GA-GA-X₍ₙ₎ (I)

wherein

ASL is an aromatic stacking ligand comprising an aromatic group, each GA is independently an amino acid or a derivative thereof, X is an amino acid or a derivative thereof, n is an integer from 0 to 3, and wherein the peptide derivatives form a gel in the presence of crosslinking cations. n is preferably 0, 1 or 2, more preferably 0 or 1 and is 0 (i.e. X is absent) in further preferred embodiments.

Suitable options for the ASL are known from the literature. Examples include Fmoc, CBz, an aromatic amino acid, and derivatives thereof.

In more preferred embodiments the peptide derivatives are of formula Ia

ASL-GA1-GA2-X₍ₙ₎ (Ia)

wherein

GA1 is selected from phenylalanine (F), tyrosine (Y), tryptophan (W) and derivatives thereof,

Each GA2 and X is independently selected from

(1) neutral amino acids alanine (A), leucine (L), asparagine (N), methionine (M), cysteine (C), glutamine (Q), proline (P), glycine (G), serine (S), isoleucine (I), threonine (T), tyrosine (Y), tryptophan (W) and valine (V), positively charged amino acids arginine (R), histidine (H) and lysine (K), and negatively charged amino acids aspartic acid (D) and glutamic acid (E), and derivatives thereof, and (2) phenylalanine (F), tyrosine (Y) and tryptophan (W) and derivatives thereof, and

n is as previously defined.

The hydrogels can comprise a mixture of peptide derivatives, some of which comprise a GA2 from list (1) and some of which comprise a GA2 from list (2).

The invention disclosed herein is of application to deliver and/or support growth of cells. Accordingly, in examples the ink further comprises cells. The ink may further comprise mimetic peptides (especially a molecule such as a peptide, a modified peptide or any other molecule that biologically mimics active ligands of hormones, cytokines, enzyme substrates, viruses or other bio-molecules).

In accordance with any of the above-described printing techniques, the ink may further comprise a gelling agent such as a polyethylene glycol, polyoxyl castor oil, such as sold under the brand name Kolliphor. Another gelling agent that can be used is fragments of laminin, and in particular IKVAV. This is a 5-amino acid chain that forms a gel, and as described above can also be used as the activator, or one of two or more activators. The ink may comprise, as further specific examples, fragments of laminin, lactobionic acid, growth factors, minerals, fibronectin and/or integrin. Other additional agents include alginate, typically in combination CaCl₂; Pluronic F127; DMEM media; PBS, and GrowDex®. One or more of these additional elements may be used in any of the printing techniques described above.

The ink can be used in a range of 3D printing methods and environments. For example, the printing may comprise extruding the ink (optionally containing cells and/or active agent). The printing may comprise printing a structure containing an active agent, for sustained delivery of the active agent, and/or printing a cell support structure containing cells.

Applications for structures include using sheets of gel. Accordingly the methods comprise printing a sheet of hydrogel precursor (again, optionally containing cells and/or active agent). One use is as a transdermal delivery composition, e.g. a patch.

Layers can be printed using the inks of the invention. Particular methods comprise
printing a first layer of ink,
allowing the ink to partially gel, and
printing a second layer of ink in contact with the first layer.

Further preferred methods of printing layers comprise
printing a first layer of partially gelled ink, and
printing a second layer of ink in contact with the first layer.

The methods preferably comprise printing the second layer (and subsequent layers) before the first layer (or immediately preceding layer) has completely gelled; this has been found to improve the bonding between adjacent layers and the consequent integrity of the printed whole.

After the printing of the partially gelled ink, the construct formed is preferably exposed to additional activator. This may be though printing into a bath of activator. The construct may also preferably be lowered into the bath of activator as each subsequent layer is printing, allowing for the layers to integrate prior to exposure to the activator. In another preferred method, the printed construct is sprayed with, or otherwise exposed to additional activator. A combination of these methods may also be used, e.g. the construct is sprayed with additional activator and then placed in a bath of activator, or placed briefly in an bath of activator and subsequently sprayed with the activator.

Multi-layered structures can be printed with different layers printed onto each other.

These multilayer structures can be exposed to additional activator as set out above.

The ink formulations and methods described above may also be used with these methods.

The invention disclosed herein is also of application to deliver active agents. Accordingly, in preferred examples and as described in more details in relation to further compositions the ink further comprises an active agent.

As described widely herein, variation of gel components and concentrations can result in gels having a range of stiffness values. Suitably, the gel has a stiffness of from 100 Pa to 100 kPa. For most applications described herein, peptide concentration and choice yields a gel having a stiffness that is 500 Pa or greater, preferably 1 kPa or greater, or more preferably 2 kPa or greater. 3D printing and drug delivery tend to require slightly stiffer gels. More generally, the gels have a stiffness of 5 kPa or greater, preferably 10 kPa or greater, more preferably 20 kPa or greater. Rheology testing of stiffness can be carried out using convention methods, e.g. atomic force microscopy (AFM) according to standard techniques and manufacturer's instructions (e.g. Veeco Instrucments, Inc, Malvern Instruments Ltd).

In specific embodiments of the invention using peptides of formula ASL-GA1-GA2-X₍ₙ₎, GA2 is an amino acid selected from R, H and K. Gels based on using these positively charged amino acids showed high levels of stiffness even in the absence of additional components such as cations, yielding stiffness values of 10 kPa and higher and 20 kPa and higher for peptide concentrations of 20 mM-30 mM.

In further specific embodiments using peptides of formula ASL-GA1-GA2-X₍ₙ₎, GA2 is an amino acid selected from D and E. Gels of the invention, based upon these negatively charged amino acids, yielded gels having acceptable stiffness, generally at similar levels to or of increased stiffness compared to a reference hydrogel (also useful in the present invention) which is a combination of Fmoc-FF with Fmoc-S in a ratio of 1:1. Of note, however, is that gels of these specific embodiments, based on negatively charged amino acids, are susceptible to having their gel stiffness increased markedly by incorporation of cations into the composition, e.g. via addition of cation containing aqueous solutions to a pre-gelation mixture. As the individual stiffness requirements for the wide range of cell types that are to be cultured or drugs to be delivered varies so greatly it was not hitherto practicable to provide a range of hydrogels that accommodate for each individual cell/drug its optimum gel stiffness requirements. By using gels of the invention, especially those incorporating negatively charged amino acids, variation of the stiffness in the final gel can be achieved by variation of peptide and/or ion, especially cation, concentration, thus improving the usability and flexibility of the gel system of the invention.

One advantage of the invention is the ability to produce stiff gels for use in 3D printing without having to use an excessively high peptide concentration. It is preferred that, in preparing compositions and gels of the invention the concentration of the peptide derivatives in the final gel is from about 5 mM to about 100 mM, more preferably 10 mM or higher, preferably up to about 70 mM, or preferably up to about 50 mM. In specific examples, gels based on peptide concentrations of approximately 20 mM and 30 mM both gave gels of high stiffness, thus without using excessively high peptide concentrations.

Tuning the properties of the gel prevents unacceptably high stiffness. Nevertheless, it is possible using the invention to produce gels having exceptionally high stiffness. Generally, the gels have a stiffness of up to about 70 kPa, preferably up to about 50 kPa, more preferably up to about 40 kPa.

Further provided by the invention is printer ink per se, for use in 3D printing applications and comprising hydrogel precursor, wherein the hydrogel precursor comprises a plurality of peptide derivatives and forms a gel in contact with a cross-linking agent or activator. The ink preferably further comprises cells and/or active agent and/or cross-linking agent as elsewhere described herein, and preferred peptides for the ink are as described elsewhere herein.

When discussing gels, stiffness (kPa) is the parameter typically used to describe the structural integrity of the scaffold. When discussing printer ink (for use in 3D printing applications and comprising hydrogel and/or precursor) the analogous parameter for the liquid form during the printing process is viscosity (cP=mPa.s). In the present invention, the viscosities of the pre-gels (comprising peptides but substantially free of cations) typically range from 1 to 250 cP, preferably from 1 to 150 cP; this compares favourably to viscosities of alginate, ranging from 300-30,000 cP, and water—just under 1 cP. The viscosities of partially gelled inks, for example when these contain some activator and further activator is needed to complete the gelling, is suitably 100 cP or more, more suitably 150 cP or more, still more suitably 200 cP or more, preferably 250 cP or more and more preferably 300 cP or more. In examples below, partially gelled inks had initial viscosities of 250-300 cP or greater. A high value for viscosity corresponds to a thick and slow moving liquid whereas a low viscosity corresponds to a thin and fast moving liquid. An advantage of our low viscosity pre-gels is the ease of printing using the ink. A further advantage in use is that combination with activator, preferably cations, leads to subsequent crosslinking (or further crosslinking) and setting of the gel; starting from a lower viscosity can enable printing using the setting ink, or partially gelled ink, before it has reached too high a viscosity for use in printers. As established elsewhere, gels of the invention are found nevertheless to set into stiff gels after crosslinking.

Still further provided by the invention are compositions comprising hydrogel precursor and active agent for use in sustained release delivery of the active agent, wherein the hydrogel precursor comprises a plurality of peptide derivatives and forms a gel in contact with a cross-linking agent.

Also provided herein are methods using the sustained delivery compositions of the invention. Thus the invention provides a method of sustained delivery of an active agent to a patient, comprising administering to the patient a hydrogel or precursor containing the active agent, wherein the hydrogel or precursor comprises a plurality of peptide derivatives and forms a gel in contact with a cross-linking agent.

The hydrogel may further comprise cells and/or cross-linking agent as elsewhere described herein, and preferred peptides for the gels are as described elsewhere herein.

Delivery of the compositions is possible by various routes, including by injection of hydrogel precursor into a patient wherein the hydrogel gels in situ and by surgical insertion of gelled hydrogel into a patient.

Apparatus for creating a cell growth support structure is also provided by the invention, comprising a container of printer ink according to the invention and as elsewhere described herein, linked to or comprised within a printer, e.g. an extruder capable of extruding the ink.

The apparatus may further comprise a container of activator, linked to an extruder capable of extruding the agent so that is contacts extruded precursor.

Particular embodiments of the invention are directed at using hydrogels as cell support structures, that is to say as matrices within which cells can grow and proliferate and optionally form multicellular structures, organelles and/or organs or parts thereof. Accordingly, the invention provides a method of forming a cell growth support structure from or comprising at least one hydrogel, comprising extruding a hydrogel precursor to enable hydrogel formation.

As described below in examples, there is provided a 3-dimensional (3D) structure or matrix that supports cell growth within or attached to the structure or which can be used for drug delivery. Suitably the structure has sufficient rigidity to provide a 3D format for cell growth. The structure may subsequently have cells added. Alternatively, the extruded hydrogel may contain one or more cells which may then grow in the hydrogel after extrusion and curing of the hydrogel.

The methods preferably comprise extruding the precursor (in non-gelled or partially gelled form) and allowing or causing it to cure: converting the more fluid precursor into a more solid hydrogel. Layers of precursor are preferably added one by one, building into the end product. Each successive layer is preferably added to partially or substantially cured hydrogel, or hydrogel that has cured enough (though possibly not yet completely) to bear the weight of and not be overly distorted by the next deposited layer, yet is able to form a cohesive gel with the subsequent layer.

In accordance with the process, precursor is extruded so as to enable gel formation. Curing or setting of precursor into a gel can be caused by combination/contact of precursor with the relevant curing agent. This is typically one or more cations, and may also be referred to as an activator.

The hydrogel precursor may be extruded and subsequently combined with the agent—for example extruded into a solution in which the hydrogel will form. This is a simple and convenient method as the equipment can operate with a single extrusion outlet linked to a supply of precursor. In examples below curing agent, e.g. cations, are present in the solution.

Alternatively, the hydrogel precursor may be extruded in combination with a hydrogel activator, which causes hydrogel formation. The two elements, namely the hydrogel precursor and the activator may be co-extruded or extruded sequentially. Prior to extrusion the precursor is stored and/or maintained separate from the activator and the two combined only shortly before extrusion.

In examples below, precursor and curing agent are extruded via a single outlet, and hence the gel is extruded in a form that has not yet gelled but is in the process of gelling or is just about to form a gel. The extrusion preferable takes place before the gel is formed to an extent that might block the extruder.

In another aspect, the invention provides a method of forming a 3D cell growth support structure from or comprising at least one hydrogel, comprising co-extruding a hydrogel precursor and a curing agent. As per examples below, the precursor preferably comprises one or more peptides and the agent comprises one or more cations.

In use, this mixing of the hydrogel precursor and the cation(s) causes rapid crosslinking of the peptides and gel formation. Concentration of both components can be adjusted and will affect this process, but very suitably this occurs essentially instantaneously upon co-extrusion.

The proportions of hydrogel precursor and activator may be such that full gelling will occur immediately, namely upon co-extrusion. In equally preferred alternatives, the hydrogel precursor and activator may be such that partial gelling will occur immediately during co-extrusion, and the construct formed can be exposed to additional activator to complete the gelling process. This can preferably result in the formation of a stiffer gel after exposure to the additional activator. The additional activator may be applied as described above, namely by placement in a bath of additional activator, or spraying. The additional activator may be the same or a different activator.

Hydrogel precursors in general are suitable for the invention; they may be single peptides or combinations of peptides. The peptides may incorporate an aromatic stacking ligand such as Fmoc or Cbz, or may include aromatic amino acids. They may be as described in general in the hydrogel art, including as referenced herein. Such suitable hydrogel precursors are described in GB1516421.2 (currently unpublished) and WO 2016/055810. Suitable tripeptides are also described in WO 2016/055810.

Examples of peptides that may be used alone specifically include Fmoc-FF; Fmoc-S; KYF, KFD, GGG, PFF, KFF, KYW, KYY, and FFD. Examples of combinations of peptides include Fmoc-FF with Fmoc-FD, Fmoc-FF with Fmoc-FE, Fmoc-FF with Fmoc-FK and F-moc-FF with Fmoc-S. Where combinations of peptides are used, different proportions of each constituent may be used to vary the ultimate stiffness of the hydrogel produced. A particularly preferred combination comprises Fmoc-FF with Fmoc-S. While approximately a 1:1 ratio may be used, varying the ratio can also be used to vary the stiffness of the gel.

In other embodiments, the proportion of precursor hydrogels to cations may be used to determine the stiffness of the final hydrogel produced. This may be achieved by varying the concentration of the solutions, or by varying the ratio of the solutions mixed together.

While the mixture of the solutions (precursor-containing and agent-containing) will typically be about 1:1, in some embodiments the ratio may be between 1:3 and 10:1 of hydrogel precursor to agent, e.g. cation solution. More typically the ratio will be between 1:2 and 4:1 and preferably will be from 1:1 to 3:1.

The co-extrusion may be into water or a buffer solution suitable for cell growth. Once the hydrogel has been formed it may be removed from the water and transferred to a plate for combination with cells.

The cations may be monovalent, divalent or trivalent. Na⁺ can be used, e.g. within a buffer such as PBS. The cations may be calcium ions, for example in the form of a calcium salt, e.g. chloride. Alternatively, calcium bromide or calcium carbonate may be used. Magnesium cations may be used, for example as magnesium chloride or magnesium bromide. In a further alternative the cations may be of iron, for example iron (III) bromide or iron (II) sulphate.

The extruder provides a controlled method of delivering the ungelled precursor, put another way, a controlled method of printing using the precursor as an ink. There is generally a high pressure area where ink is located prior to printing and a low pressure area, to which ink is delivered/extruded via an outlet, generally a nozzle. The nature of the nozzle and the process for delivering precursor at a particular rate or width or shape of output is not the subject of this invention.

In its simplest form the extruder may be a double barrel syringe. Hydrogel precursor may be loaded into one barrel of the syringe and a solution containing cations may be loaded into the other barrel. Urging of the plungers into the syringes discharges the hydrogel precursor and the cation solution such that they then come into contact, causing mixing and gelation. Using this method approximately equal volumes of the hydrogel precursor and the cation solution will be dispensed. As such is it preferred to prepare the solutions in appropriate concentration for gelation to occur on a 1:1 solution mixture.

In an alternative, and also simple, system that illustrates the invention, a 3-way connector may be used with two containers (e.g. syringes) attached and one outlet. As before hydrogel precursor may be loaded into one and a cation solution loaded into the other. Dispensing from the containers (e.g. depression of the plungers on the syringes) will dispense the solutions, which mix and more or less immediately form a hydrogel exiting the outlet. The plungers to both syringes may be pressurised to the same amount, dispensing a 1:1 ratio of the solutions. Alternatively, a higher pressure may be applied to one of the plungers which may results in a 2:1 or 3:1 mixture of the components.

In a further specific alternative the solutions may be loaded into a double chamber printer for dispensing through a nozzle. The quantities and thus relative proportions of the solutions may be carefully controlled to ensure that the correct proportions of the solutions are dispensed to optimise gel formation and to vary the stiffness of the gel.

Preferably the bio-ink may be prepared, including the peptide derivatives and activator, and other elements, and transferred to the printer cartridge. The bio-ink will start to gel on forming, and printing can occur prior to full gelation. Full gelation will take place following printing.

Apparatus for creating a cell growth support structure (especially as described elsewhere herein) is further provided by the invention, the apparatus comprising a container of hydrogel precursor, linked to an extruder capable of extruding the precursor. This is the “3D printer” loaded with hydrogel precursor.

Preferred apparatus are those further comprising a container of agent that cures the precursor, linked to a extruder capable of extruding the agent so that is contacts extruded precursor. In this way, the printer is suitable for co-extrusion as described e.g. above. Other printers can extrude precursor into solution containing curing agent.

The extruder suitably comprises a moveable nozzle. Separately, the extruder suitably comprises a nozzle with variable output dimensions. Hence, width and shape of the layers can be varied during “printing” of the cell growth support structure.

The compositions of the invention can be provided in a dry form. By way of example, the composition can be in the form of a powder or lyophilised preparation. In one use water is added to the dry composition to yield a pre-gelation mixture, which in turn is combined with cross linker, e.g. cations in solution, to yield a gel. For example, the powder is reconstituted with cation solution then promptly used for printing before it gels fully. Alternatively, the dry form is combined in a single step with cations in solution to yield a mixture which itself spontaneously forms a gel. Dry formats of the composition can be prepared by subjecting an aqueous solution of peptides/derivatives to standard drying techniques. As one such example, a pre-gelation mixture is freeze-dried to yield a dry form of the invention. Dry powder composition are preferred due to their ease of storage and transportation.

Compositions of the invention that are not provided as the final gel composition are provided dry or as pre-gelation mixtures. These can be marked to show the suitable amount of cross linker, e.g. in aqueous carrier, to add to achieve the desired gel. In embodiments of the invention, therefore, the composition is provided in dry form or in the form of a pre-gelation mixture, for combination with a predetermined amount of cross linker, such as a volume of aqueous carrier, so that in the resultant gel the concentration of the peptide derivatives is from 5 mM to 100 mM, preferably 10 mM to 50 mM. Products in this form can hence be marked to indicate the gel properties obtained when the dry form or pre-dilation mixture is combined with the given carrier amounts. The products may also be marked to indicate how the gel properties will vary according to other components of the carrier such as ions, especially cations.

Gels used in the invention preferably spontaneously form as 3 dimensional gels with measurable stiffness, under conditions of physiological pH, generally at pH from about 6 to about 9, more suitably at pH in the range from about pH 6 to 8, e.g. pH 7 to 8, human body pH being typically about 7.4. When provided in, say, powdered form addition of a predetermined amount of known culture media containing known ions, supplements etc. yields a solution having a pH in the above range; the gel then forms and stiffens spontaneously. Alternatively, a pre-gelation mixture can be provided in liquid form; addition of a predetermined amount of media then yields a solution with pH in the gelling range above, and the gel forms.

During gel formation, fibres are formed within the gel by stacking of adjacent peptides into straight and, in some cases, branched fibres. The formation of this fibre-containing matrix is facilitated by the presence of the aromatic stacking ligands, suitably as described in US 2013/0338084 and U.S. Pat. No. 8,420,605. Examples include Fmoc, Cbz and derivatives thereof. Aromatic stacking interactions are recognised and well described in the scientific literature and arise from the attractive force between the pi-electron clouds in adjacent/neighbouring aromatic groups. The aromatic stacking can be affected by various factors including pH, and as described elsewhere herein the forces are strong enough so that present gels can be formed at physiological pH. In the invention, fibres formed by these stacking forces between e.g. adjacent π-electrons in Fmoc groups or other ASLs create the stiff gels described. Preferred embodiments below use Fmoc or a derivative thereof. In specific examples, described in more detail below, the ASL is Fmoc.

Compositions of the invention are suitable for growth of animal, especially mammalian cells and preferably human cells. Different types of cell require a different stiffness in the scaffold for optimum growth. For example, brain cells require a relatively soft gel, while bone cells require a much stiffer gel. However because the stiffness of the gel can be fine-tuned (by altering the ratio of hydrogel precursor and activator for example), the scaffold can be adjusted to accommodate for almost any type of cell. Preferably, such hydrogels form a sufficiently stiff gel to support cell growth and do so under physiological pH. In addition, it is advantageous for the gel to be optically transparent to aid monitoring of the cells, not be too concentrated (as low concentration of gel components or large pore sizes between gel components can be beneficial for cell growth) and have sufficient longevity for the growth required.

Active agents for sustained delivery can be widely selected for the present invention. The term “active agent” intends the active agent(s) optionally in combination with pharmaceutically acceptable carriers and, optionally additional ingredients such as antioxidants, stabilizing agents, permeation enhancers, etc. The active agent delivery compositions of the invention find use where the prolonged and controlled delivery of an active agent is desired. They especially find use when access to a deposition site is restricted and is facilitated by the ability to deliver the composition by injection of a pregel, e.g. subcutaneous, intraparenteral, etc., which then gels in situ to form a stiff gel providing for sustained delivery.

Suitable agents include but are not limited to pharmacologically active peptides, polypeptides and proteins, genes and gene products, other gene therapy agents, other biologics and other small molecules. The polypeptides may include but are not limited to growth hormone, somatotropin analogues, somatomedin-C, Gonadotropic releasing hormone, follicle stimulating hormone, luteinizing hormone, LHRH, LHRH analogues such as leuprolide, nafarelin and goserelin, LHRH agonists and antagonists, growth hormone releasing factor, calcitonin, colchicine, gonadotropins such as chorionic gonadotropin, oxytocin, octreotide, somatotropin plus an amino add, vasopressin, adrenocorticotrophic hormone, epidermal growth factor, prolactin, somatostatin, somatotropin plus a protein, cosyntropin, lypressin, polypeptides such as thyrotropin releasing hormone, thyroid stimulation hormone, secretin, pancreozymin, enkephalin, glucagon, endocrine agents secreted internally and distributed by way of the bloodstream, and the like. Further agents that may be delivered include aiantitrypsin, factor VIII, factor IX and other coagulation factors, insulin and other peptide hormones, adrenal cortical stimulating hormone, thyroid stimulating hormone and other pituitary hormones, interferon α, β, and δ, erythropoietin, growth factors such as GCSF, GMCSF, insulin-like growth factor 1, tissue plasminogen activator, CD4, dDAVP, interleukin-1 receptor antagonist, tumor necrosis factor, pancreatic enzymes, lactase, cytokines, interleukin-1 receptor antagonist, interleukin-2, tumor necrosis factor receptor, tumor suppresser proteins, cytotoxic proteins, and recombinant antibodies and antibody fragments, and the like.

Active agents useful herein are for the treatment of a variety of conditions including but not limited to hemophilia and other blood disorders, growth disorders, diabetes, leukemia, hepatitis, renal failure, HIV infection, hereditary diseases such as cerbrosidase deficiency and adenosine deaminase deficiency, hypertension, inflammation, septic shock, autoimmune diseases such as multiple sclerosis, Graves disease, systemic lupus erythematosus and rheumatoid arthritis, shock and wasting disorders, cystic fibrosis, lactose intolerance, Crohn's diseases, inflammatory bowel disease, gastrointestinal and other cancers.

The active agents may be anhydrous or aqueous solutions, suspensions or complexes with pharmaceutically acceptable vehicles or carriers. The active agents may be in various forms, such as uncharged molecules, components of molecular complexes or pharmacologically acceptable salts. Also, simple derivatives of the agents (such as prodrugs, ethers, esters, amides, etc.) which are easily hydrolyzed by body pH, enzymes, etc., can be employed.

It is to be understood that more than one active agent may be incorporated into the compositions and that the use of the term “agent” refers also to such multi-component formulations. The compositions find use, for example, in humans or other animals. The environment of use is a fluid environment and can comprise any subcutaneous position or body cavity, such as the peritoneum or uterus, and may or may not be equivalent to the point of ultimate delivery of the active agent formulation. A single gelled composition can be administered to a subject during a therapeutic program. The gels are designed to remain gelled during a predetermined administration period and to break down over time, leaving substantially no residue; hence, they do not need to be removed but essentially are biodegradable in situ.

Compositions of the present invention are useful for the sustained delivery of poorly water soluble drugs, e.g. having solubilities of less than 10 mg/mL at ambient temperatures. Examples of these hydrophobic drugs include anticancer agents, anti-inflammatory agents, antifungal agents, anti-emetics, antihypertensive agents, sex hormones, and steroids. Typical examples of these hydrophobic drugs are: anticancer agents such as paclitaxel, docetaxel, camptothecin, doxorubicin, daunomycin, cisplatin, 5-fluorouracil, mitomycin, methotrexate, and etoposide; anti-inflammatory agents such as indomethacin, ibuprofen, ketoprofen, flubiprofen, dichlofenac, piroxicam, tenoxicam, naproxen, aspirin, and acetaminophen; antifungal agents such as itraconazole, ketoconazole and amphotericin; sex hormones such as testosterone, estrogen, progesterone, and estradiol; steroids such as dexamethasone, prednisolone, betamethasone, triamcinolone acetonide and hydrocortisone; antihypertensive agents such as captopril, ramipril, terazosin, minoxidil, and parazosin; antiemetics such as ondansetron and granisetron; antibiotics such as metronidazole, and fusidic acid; cyclosporines; prostaglandins; and biphenyl dimethyl dicarboxylic acid.

Gels containing positively charged groups can be used for active agents that are negatively charged, and vice versa. The ability to tune gel chemistry and stiffness offers gels suitable for substantially all active agents.

Sustained drug delivery achievable using the gels of the invention has extended in an example beyond 7 days. In general, however, the gels can deliver active agents in a sustained profile over 2 days or more, 3 days or more, preferably 4 days or more, 5 days or more, or 6 days or more. The delivery period can depend also upon therapy regime selected for that particular patient and/or active agent. Gel compositions can be adjusted e.g. by increasing peptide concentration or increasing cross linker concentration or both to provide for stiffer gels with more prolonged release profiles.

### Specific Embodiments

In specific embodiments of the invention optional and preferred features of the invention are combined into particular compositions. The gels are used for 3D printing or as cell support structures or for sustained active agent delivery.

Selected specific embodiments comprise gels composed of di-peptides linked to an ASL, cross linked by divalent cations having a stiffness of 5 kPa or higher at a pH of 6-8.

Further selected specific embodiments comprise gels composed of mixtures of di-peptides linked to an ASL, cross linked by calcium and/or magnesium ions having a stiffness of 5 kPa or higher at a pH of 6-8, with stiffer embodiments having a stiffness of 10 kPa or higher.

In one set of specific embodiments the gel comprises a mixture (e.g. of from 1:5 to 5:1, preferably from 1:2 to 2:1, more preferably approximately 1:1) of

(a) Fmoc-FF and one or more of:

(b) Fmoc-S (i.e. yielding a mixture of a dipeptide and a mono-peptide), Fmoc-FF (i.e. the gel is pure Fmoc-FF), Fmoc-FS, Fmoc-FR, Fmoc-FH, Fmoc-FK, Fmoc-FD or Fmoc-FE. These form gels of stiffness 5 kPa or greater, preferably 10 kPa or higher at pH 6-8.

In a specific embodiment of 3D printing, there is provided a method of printing, comprising:
a. preparing ink comprising a mixture of hydrogel precursor and activator in non-gelled form,
b. printing the ink onto a substrate, and
c. allowing the ink to gel,
wherein the hydrogel precursor comprises a plurality of peptide derivatives composed of di-peptides linked to an ASL and the activator comprises a cross-linking cation selected from calcium and magnesium and mixtures thereof, and
wherein the ink when gelled has a stiffness of 5 kPa or greater.

In a further specific embodiment of 3D printing, there is similarly provided a method of printing, comprising
a. preparing ink comprising a hydrogel precursor in non-gelled form,
b. printing the ink so as to contact it with activator, and
c. allowing the ink to gel,
wherein the hydrogel precursor comprises a plurality of peptide derivatives composed of di-peptides linked to an ASL and the activator comprises a cross-linking agent selected from calcium and magnesium and mixtures thereof, and
wherein the ink when gelled has a stiffness of 5 kPa or greater.

In a further specific embodiment of 3D printing, there is provided a method of printing, comprising
a. preparing ink comprising a hydrogel precursor in partially gelled form,
b. printing the ink onto a substrate, and
c. allowing the ink to fully gel,

wherein the hydrogel precursor comprises a plurality of peptides or peptide derivatives, and an activator, optionally further including a further gelling agent, such as a polyoxyl castor oil or laminin fragment, and wherein the activator is a cation, such as a metal cation, or the cationic part of a peptide zwitterion, such as IKVAV.

The ink may also be printed directly into a solution containing a further activator, for example cell culture media such as DMEM.

In a still further specific embodiment of 3D printing, there is provided a method of printing, comprising
a. preparing ink comprising a hydrogel precursor and activator in partially gelled form,
b. printing the ink onto a substrate,
c. combining the ink with further activator, and
d. allowing the ink to fully gel,

wherein the hydrogel precursor comprises a plurality of peptides or peptide derivatives. The combining of c. is preferably achieved by printing into a solution comprising the activator.

Specific printer inks per se, for use in 3D printing applications, comprise (1) hydrogel precursor, wherein the hydrogel precursor comprises a plurality of peptide derivatives composed of di-peptides linked to an ASL and forms a gel in contact with a cross-linking agent selected from calcium and magnesium and mixtures thereof, and (2) cells and/or active agent, wherein the inks when gelled have a stiffness of 5 kPa or greater. Optionally the compositions include the cations.

Specific compositions, for use in sustained active agent delivery applications, comprise (1) hydrogel precursor, wherein the hydrogel precursor comprises a plurality of peptide derivatives composed of di-peptides linked to an ASL and forms a gel in contact with a cross-linking agent selected from calcium and magnesium and mixtures thereof, and (2) active agent, wherein the inks when gelled have a stiffness of 5 kPa or greater. Optionally the compositions include the cations.

Preferred embodiments of the inks and compositions apparatus comprise optional and preferred embodiments of precursor and curing agent as described elsewhere herein.

### Advantages

In use, various advantages may be realised using the invention. Known hydrogels are based on alginates, which have various problems: they require high calcium concentrations e.g. 100 mmol and higher, whereas such concentrations can be detrimental to cell growth. The gels herein do not use these high concentrations and are hence more amenable to cell growth uses. On the other hand the alginate gels are generally not sufficiently stiff to be useful in 3D printing or sustained drug delivery unless very high calcium levels are used. The peptide gels herein can be made stiffer and at lower cation concentration e.g. about 20-30 mmol.

Known alginate gels have routinely to be blended with collagen to give them desirable properties; this does not apply to the present peptide-based gels. Known alginate gels tend to be elastic; cells do not grow well in elastic gels; again, this problem is overcome in the peptide gels herein.

Being derived from seaweed, alginate is inconsistent in quality and difficult to make as a GMP product; this is not the case for the present peptide gels. Alginate gels are found not to be functional for cells and need to have extra components added to make it functional, i.e. to enable cell growth. Good cell growth is achieved in the present peptide gels, confirming functionality without the need for additional components and also confirming the gels are biocompatible and not toxic to cell growth.

A further advantage is that the invention enables injection of the drug delivery structure. 3D printing during surgery is also possible, directly onto the patient.

Fmoc as used in certain gels is found to be anti-inflammatory and hence the gels/structures etc. can incorporate this useful property.

### SPECIFIC DESCRIPTION

To help understanding of the invention, specific embodiments thereof will now be described by way of example and with reference to the accompanying drawings in which:

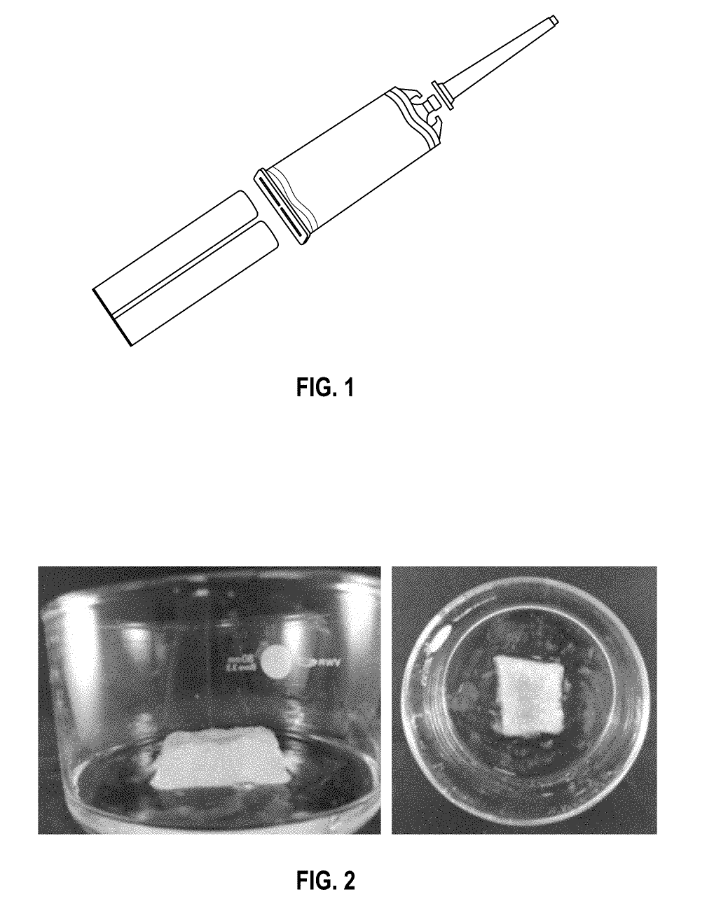

FIG. 1 shows a double barrel syringe loaded with hydrogel precursor and calcium chloride solution for simultaneous dispersion;

FIG. 2 shows photographs of a gel formed from Run 5;

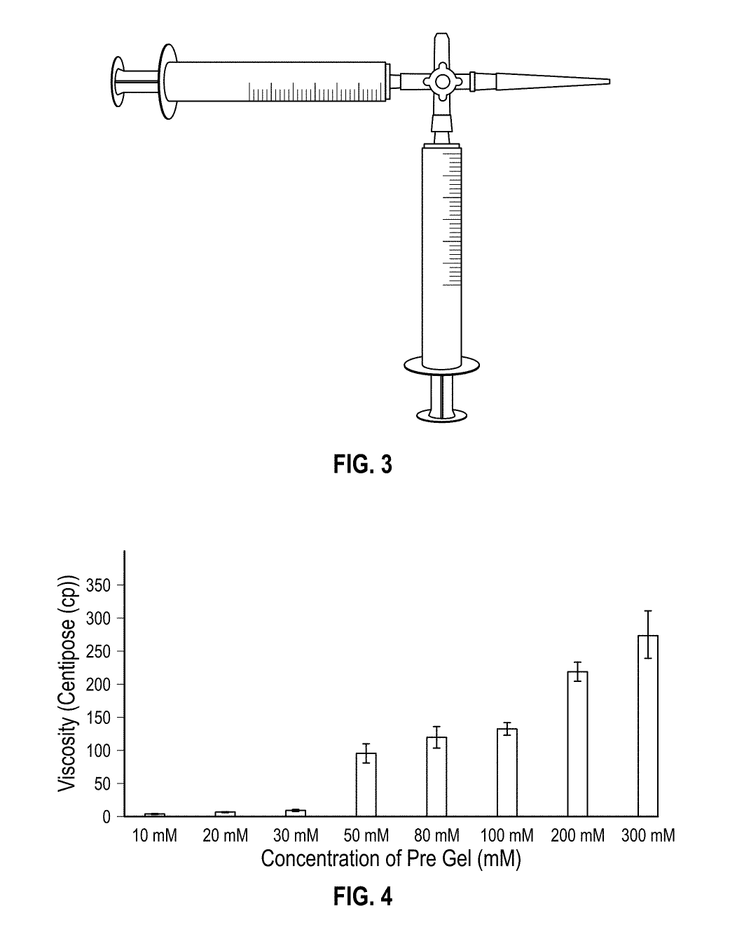

FIG. 3 shows a photograph of a three-way connector with two syringes attached;

FIG. 4 is a graph showing the viscosity of pre-gels prepared from lyophilised powder;

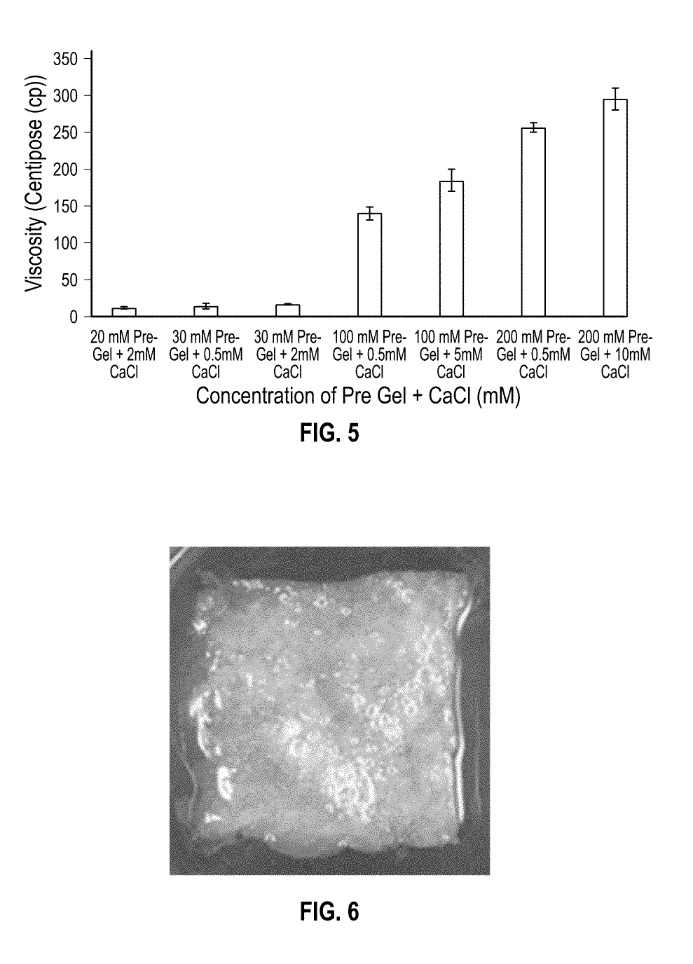

FIG. 5 is a graph showing the viscosity of pre-gels prepared from lyophilised powder and calcium chloride solution;

FIG. 6 shows the hydrogel from Run 10;

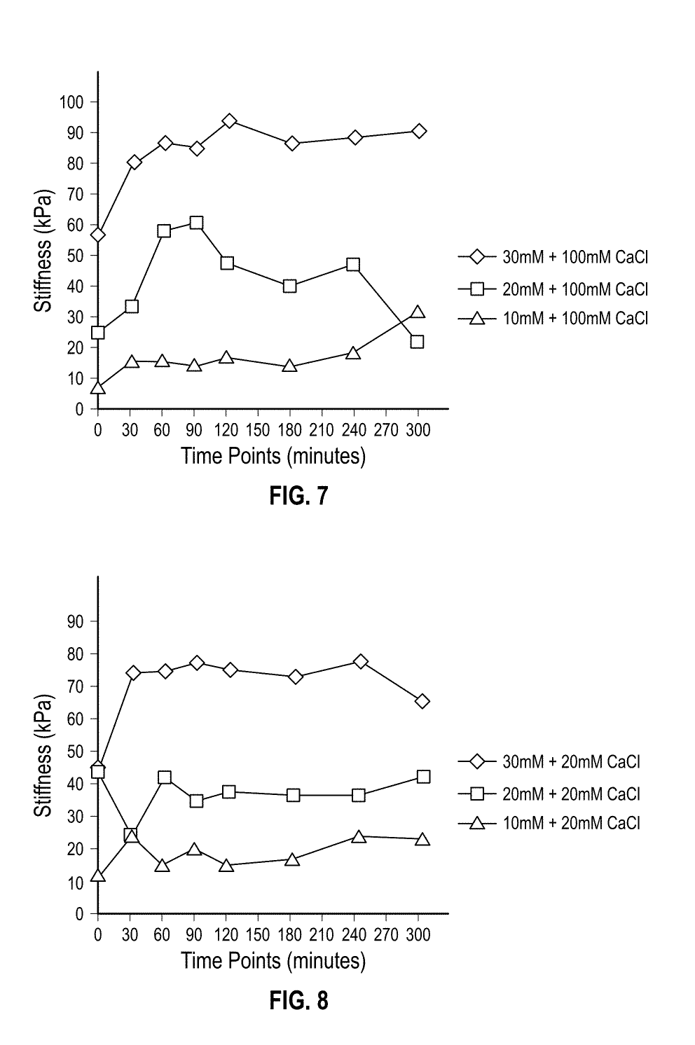

FIG. 7 is a graph showing stiffness differences (kPa) between the three gel types (soft, soft-to-firm and firm) and the stability over a short period of time;

FIG. 8 is a graph showing stiffness differences (kPa) between the three gel types (soft, soft-to-firm and firm) and the stability over a short period of time;

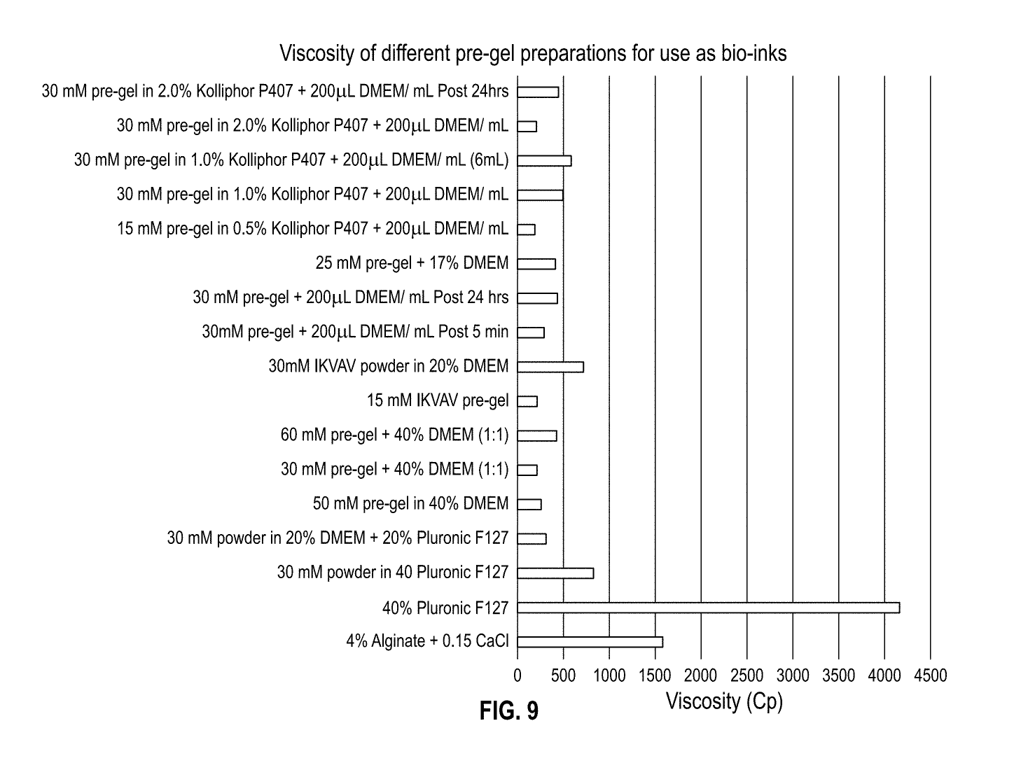

FIG. 9 is a graph showing the viscosity results of potential bio-inks prepared from different combinations of materials;

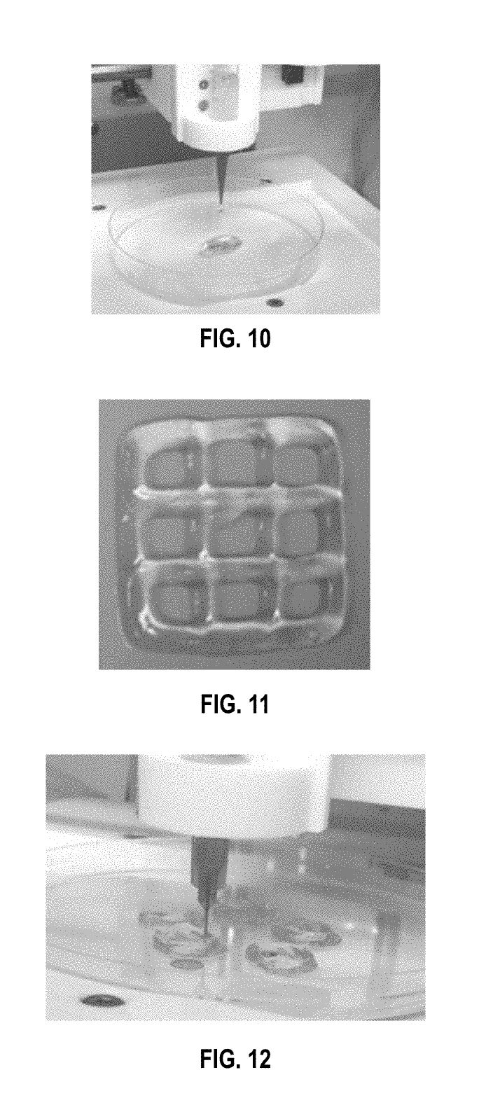

FIG. 10 shows a photograph of printing using Cellink Inkredible printer with an ink according to the invention;

FIG. 11 shows a photograph of printed gel grid structure (5 layers) from Run 14;

FIG. 12 shows a photograph of printed gel cylinder structure from Run 15;

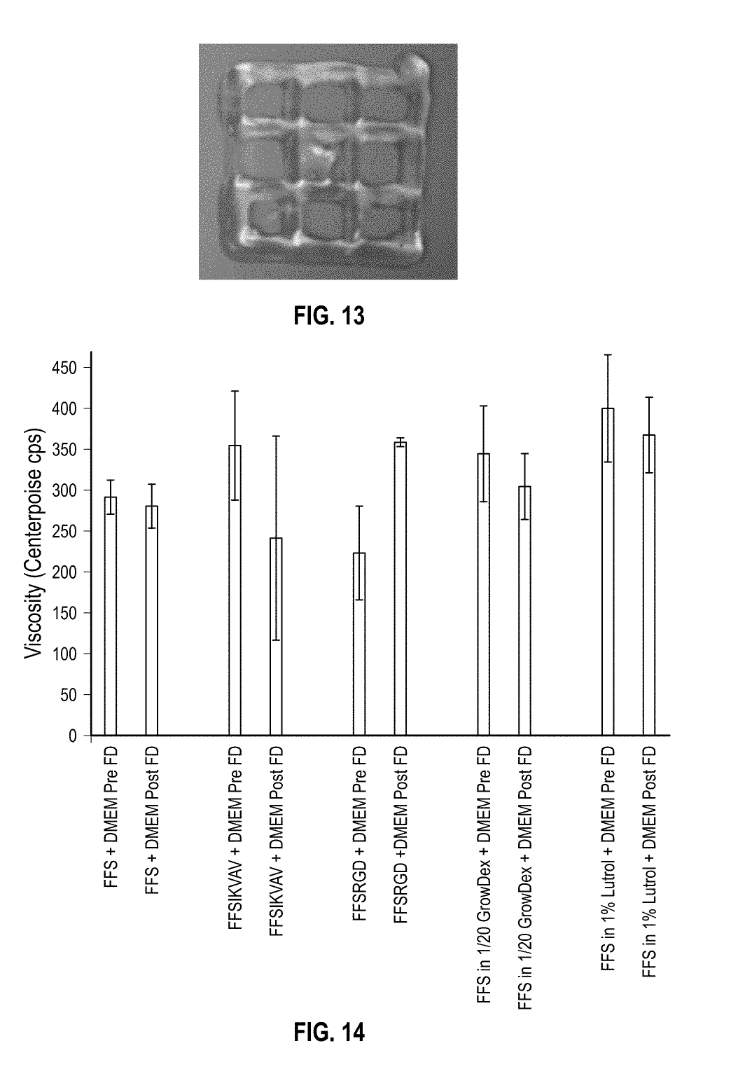

FIG. 13 shows a photograph of printed gel grid structure (5 layers) from Run 15;

FIG. 14 is a graph showing viscometry results of bio-inks according to the invention pre and post freeze drying;

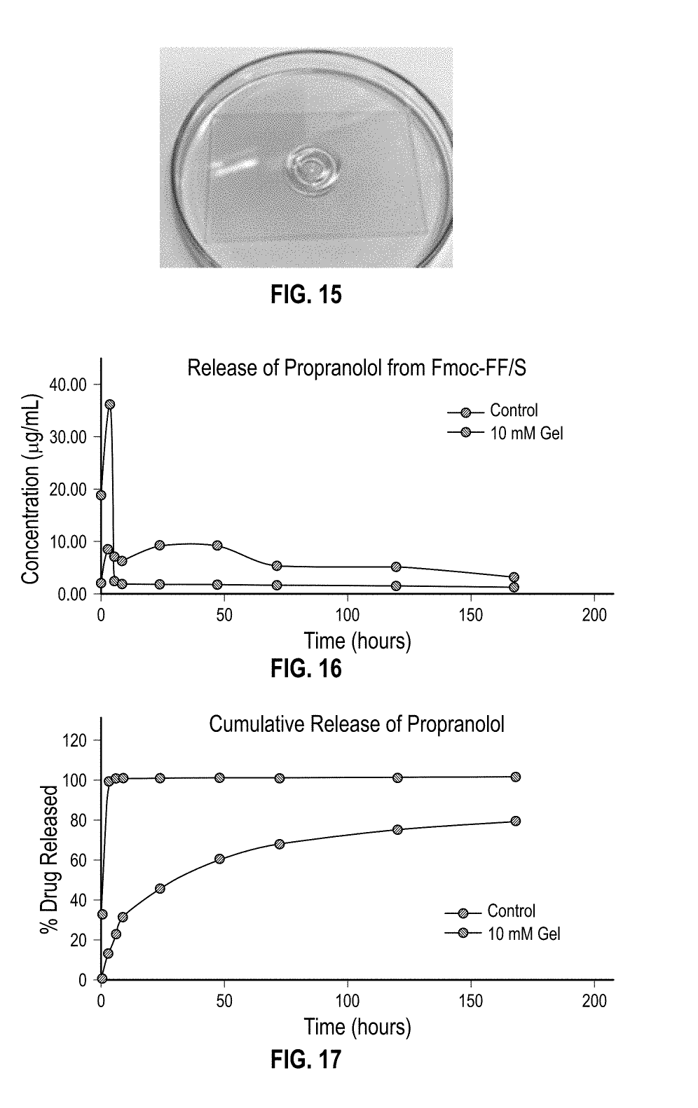

FIG. 15 is a photograph of a structure printed by the printer of FIG. 10 immediately after printing;

FIG. 16 is a graph of the release profile of Propranolol from 10 mM Fmoc-FF/S;

FIG. 17 is a graph of the cumulative release profile of Propranolol from 10 mM Fmoc-FF/S;

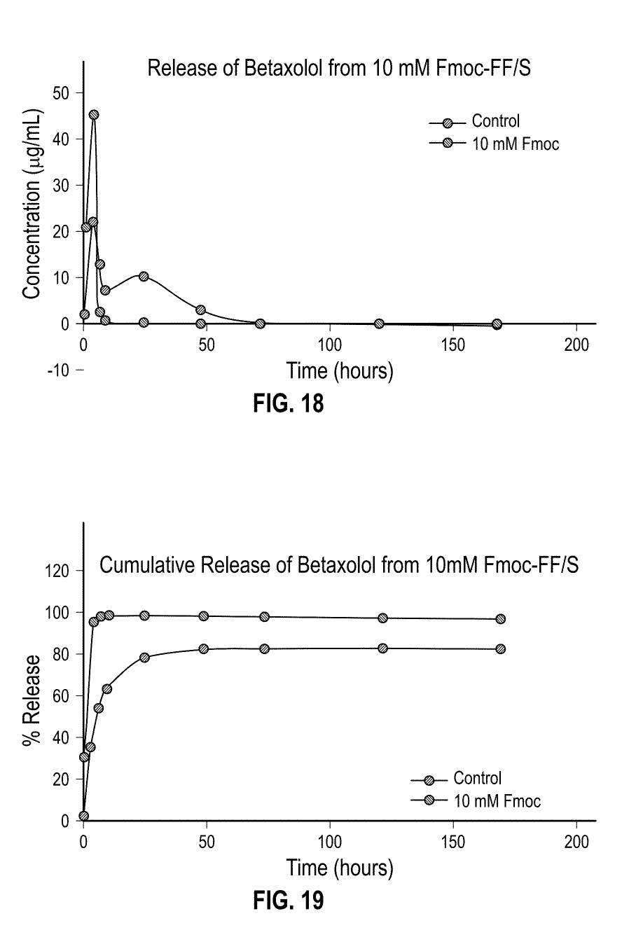

FIG. 18 is a graph of the release profile of Betaxolol from 10 mM Fmoc-FF/S;

FIG. 19 is a graph of the cumulative release profile of Betaxolol from 10 mM Fmoc-FF/S;

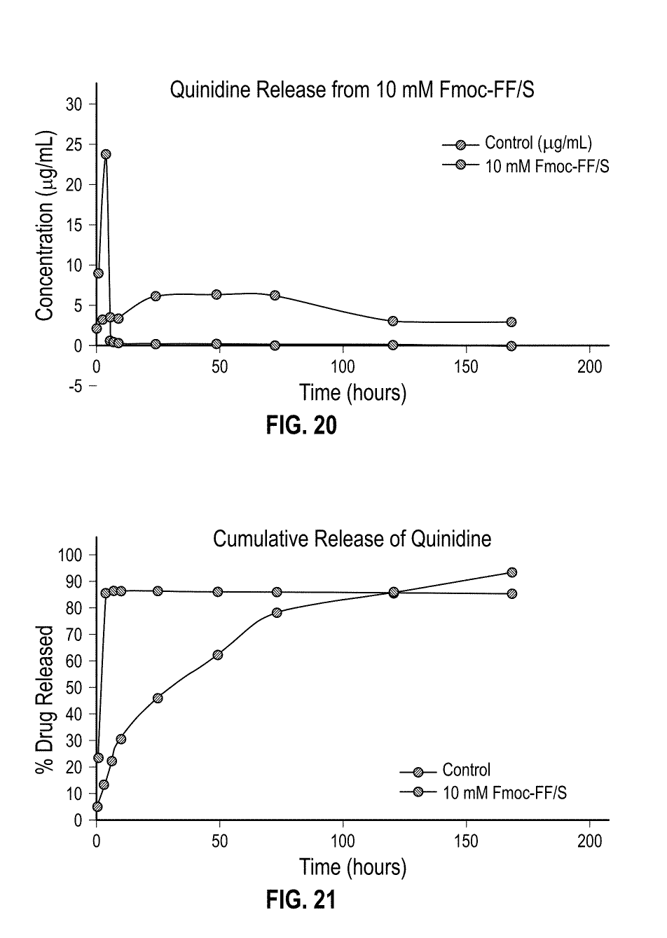

FIG. 20 is a graph of the release profile of Quinidine from 10 mM Fmoc-FF/S; and

FIG. 21 is a graph of the cumulative release profile of Quinidine from 10 mM Fmoc-FF/S.

### EXAMPLE 1

Two methods of printing using hydrogels were investigated. The first used pre-gel mixed with CaCl₂ solution to produce a partially crosslinked material which was dispensed into a concentrated CaCl₂ solution to rapidly fully gel the material in a 3D structure. The second comprised dispensing pre-gel with CaCl₂ solution via a double barrel syringe to immediately cause cross-linking and for a 3D structure to be created.

Preparation of the Solutions.

Hydrogel Precursor Solution

Fmoc-FF/S (i.e. a mixture of Fmoc-FF and Fmoc-S) lyophilised powder (batch produced using 91% pure Fmoc-FF for investigation purpose only) was weighed into a 50 mL tube, which had been tared on the balance. To obtain hydrogel precursors with concentrations of 10, 20 and 30 mM, 0.22, 0.44 and 0.66 grams were used and reconstituted in sterile water. Thorough mixing and sonication for 30 seconds was performed using the vortex and sonicator water bath. Pre-gels were stored at 4° C. until further use.

Calcium Chloride Solution

Calcium Chloride solution was prepared at 5, 20 and 100 mM concentrations by weighing out 0.055, 0.222 and 1.110 grams of calcium chloride into a beaker and making the volume up to 100 mL with sterile water. A magnetic bead and stirring platform was used to dissolve the calcium chloride by mixing for 10 minutes. The solutions were then 0.2 μm syringe filtered into a clean glass beaker and stored at 4° C. until further use.

3D Structure Formation

The 3D structures were formed using the double barrel syringe shown in FIG. 1. The rubber pistons supplied with the syringe, were attached to the plunger with adhesive so that they could be used multiple times. Hydrogel precursor was loaded into one side of the syringe and a CaCl₂ solution in the other side. The plunger with pistons attached was inserted and the syringe cap was removed so that a 200 μL tip could be placed on.

The mix of pre-gel/CaCl₂ solution used was as detailed in the following table;

TABLE 1

| | Pre-Gel | Concentration of |
|---|---|---|
| Run | Concentration (mM) | CaCl₂ (mM) |
| 1 | 10 | 20 |
| 2 | 10 | 100 |
| 3 | 20 | 20 |
| 4 | 20 | 100 |
| 5 | 30 | 20 |
| 6 | 30 | 100 |

Results:

The original experiments used pre-gel and CaCl₂ solutions incubated at 37° C., 5% CO₂ prior to use and returned to these conditions between use. The glass container with 3D structure was incubated at the same conditions (37° C., 5 % CO₂). Subsequently, the work was performed at room temperature, to demonstrate that 3D structure formation is not temperature dependent.

A cube was used to demonstrate 3D structure capabilities of the peptide gel. A section of paper was placed under the glass container with a square (˜2 cm²) drawn on it and was used as a template for the 3D cube. The 3D structure was created by moving the dispensing syringe, in effect the nozzle, from top left to bottom, across then up to top right corner of the square template into the glass container. This was repeated three times in a continuous flow so as the same volume of material was dispensed for each pre-gel and CaCl₂ solution combination.

Structures

Run 1, namely 10 mM pre-gel+20 mM CaCl₂ solution, resulted in a cube although some of the material was not attached to the main body as the gel took a few seconds (2-3 s) to form a solid gel.

Run 2, namely 10 mM pre-gel+100 mM CaCl₂ solution, resulted in a cube although some of the material was again detached from the main body as the gel look a few seconds (2-3 s) to form as a solid gel. It was also observed that the stronger CaCl₂ solution resulted in a more opaque gel material.

Runs 4 and 3, namely 20 mM pre-gel+100 mM CaCl₂ solution and 20 mM pre-gel +20 mM CaCl₂ solution respectively resulted in similar results as for the 10 mM pre-gel material but with the gel material forming slightly quicker (1-2 s).

Run 5, as shown in FIG. 5, namely 30 mM pre-gel+20 mM CaCl₂ solution, resulted in a cube with a lot less of the material not attached to the main body as the gel. The gel material formed very quickly and seemed to gel almost instantaneously to form a solid 3D cube.

Run 6, namely 30 mM pre-gel+100 mM CaCl₂ solution, resulted in a cube again with a lot less of the material not attached to the main body as the gel. The gel material formed very quickly and seemed to gel almost instantaneously to form a solid 3D cube. The stronger CaCl₂ solution resulted in a more opaque gel material.

Conclusion:

The first method of pre-setting the gel was used initially then refined as the second method (see discussion below). Note that some methods utilised also encapsulated individual cells in small volumes of gel.

The second method was successful for producing a 3D cube structure built up from many extruded layers, gelled on top of each other. With more controlled dispensing systems more defined and more complex structures were achievable. The 3D cubes made retained the cube shape and when immersed in water again retained their shape.

The invention hence provides a method of creating 3D hydrogel structures using a technique akin to 3D printing.

### EXAMPLE 2

Following on from Example 1, a second experimental procedure was devised and used to dispense pre-gel and CaCl₂ solution from two separate syringes through a 3-way connector (see FIG. 3). The two components were dispensed through the third opening on the connector via a 1 mL pipette tip, which had been attached. The gel material produced was tested for rheology over a 5 hour period to demonstrate short term stability.

Having identified the concentration of calcium chloride solution required to initiate quick gelation of the peptide derivatives, initial investigation into the mechanical properties of the structures formed under these new conditions was carried out, and an assessment of the tunability of the new bio-ink.

With regards to specific mechanical properties that were studied, initially the viscosity of the bio-ink was investigated as this is a key component in determining its compatibility with 3D bioprinting techniques.

The stiffness of the fully cross-linked printed constructs were also measured using rheology. This is an important characteristic, as not only will it have an effect on the stability of the printed structure, as successive layers of material are deposited, but the stiffness will also have an influence over the behaviour of cells incorporated into the gel, with tunability being a highly attractive feature within 3D cell culture. As gelation of these new bio-ink gels have been triggered under alternative conditions to standard cell culture protocols, the stiffness of the gels were found to be different.

Preparation of the Solutions

Fmoc-FF/S lyophilised powder (batch produced using 91% pure Fmoc-FF for investigation purpose only) was weighed into a 50 mL tube. To obtain pre-gels with concentrations of 10, 20, 30, 50, 80, 100, 200 and 300 mM, 0.22, 0.44, 0.66, 1.1, 1.76, 22, 44 and 66 grams were used and reconstituted in sterile water. Thorough mixing and sonication for 30 seconds was performed using the vortex and sonicator water bath. Pre-gels were stored at 4° C. until further use. Calcium chloride solutions were prepared at 0.5, 2, 5 and 10 mM concentrations as it was believed these concentrations would achieve partial crosslinking but not full gelation.

The viscosity results of pre-gel alone and pre-gel mixed with calcium chloride solution are detailed in the following tables;

TABLE 2

Viscosity Centipoise

| Pre-Gel Concentration (mM) | R1 | R2 | R3 | Average |
|---|---|---|---|---|
| 10 | 5.0 | 4.2 | 3.5 | 4.2 |
| 20 | 6.8 | 7.2 | 6.8 | 6.9 |
| 30 | 10.8 | 9.9 | 9.5 | 10.1 |
| 50 | 113.1 | 910.0 | 906.0 | 98.2 |
| 80 | 116.6 | 111.0 | 140.0 | 122.5 |
| 100 | 134.0 | 131.0 | 146.0 | 137.0 |
| 200 | 213.2 | 222.0 | 239.0 | 224.8 |
| 300 | 238.7 | 296.0 | 306.0 | 280.1 |

TABLE 3

| Pre-Gel Concentration (mM) + | Viscosity Centipoise | | | |
|---|---|---|---|---|
| CaCl₂ Solution (mM) | R1 | R2 | R3 | Average |
| 20 + 2 | 14.2 | 12.7 | 12.1 | 13.0 |
| 30 + 0.5 | 18.3 | 15.9 | 10.3 | 14.8 |
| 30 + 2 | 17.6 | 17.3 | 16.7 | 17.2 |
| 100 + 0.5 | 136.3 | 134.0 | 150.0 | 140.3 |
| 100 + 5 | 179.6 | 174.0 | 202.0 | 185.3 |
| 200 + 0.5 | 253.3 | 252.5 | 263.6 | 256.5 |
| 200 + 10 | 276.3 | 294.1 | 305.7 | 292.0 |

The graphs of FIG. 4 and FIG. 5 have been prepared from this data. An increase in pre-gel concentration in turn increased the viscosity of the material. The viscosity results also demonstrated that with addition of calcium chloride solution the material becomes more viscous compared to the same concentration of pre-gel with no calcium chloride solution addition.

Using this knowledge further 3D printing experiments were conducted using the pre-gel and CaCl₂ solutions dispensed from two separate syringes through a three way connector as set out above.

Preparation of the Solutions

Pre-Gel Formation

Fmoc-FF/S lyophilised powder (batch produced using 91% pure Fmoc-FF for investigation purpose only) was weighed into a 50 mL tube. To obtain pre-gels with concentrations of 10, 20 and 30 mM, 0.22, 0.44 and 0.66 grams were used and reconstituted in sterile water. Thorough mixing and sonication for 30 seconds was performed using the vortex and sonicator water bath. Pre-gels were stored at 4° C. until further use.

Calcium Chloride Solution

Calcium Chloride solution was prepared at 20 and 100 mM concentrations by weighing out 0.222 and 1.110 grams of calcium chloride into a beaker and making the volume up to 100 mL with sterile water. A magnetic bead and stirring platform was used to dissolve the calcium chloride by mixing for 10 minutes. The solutions were then 0.2 μm syringe filtered into a clean glass beaker and stored at 4° C. until further use.

3D Structure Formation

Pre-gel mixed with a CaCl₂ solution was found to immediately form crosslinked material.

As shown in FIG. 3, a 3-way syringe connector with two syringes attached, was loaded with pre-gel in one syringe and CaCl₂ solution in the other. The plunger of the syringes were pressured at the same time so the mix of pre-gel and CaCl₂ solution was a 50:50 ratio.

The mix of pre-gel/CaCl₂ solution used was as detailed in the following Table 4:

TABLE 4

| | Pre-Gel | Concentration of CaCl₂ to |
|---|---|---|
| | Concentration | obtain Fully Cross-linked 3D |
| Run | (mM) | Structure (mM) |
| 7 | 10 | 20 |
| 8 | 10 | 100 |
| 9 | 20 | 20 |
| 10 | 20 | 100 |
| 11 | 30 | 20 |
| 12 | 30 | 100 |

Appearance

A section of paper was placed under a glass container with a square (4 cm²) drawn on it and was used as a template for the 3D cube. The 3D structure was created by moving the 4-way connector with the two syringes from top left to bottom, across then up to top right corner of the square template into the glass container. This was repeated twice in a continuous flow so as the same volume of material was dispensed for each pre-gel and CaCl₂ solution combination. Three different gel concentrations were selected 30 mM, 20 mM and 10 mM (representing firm, soft-to-firm and soft gels).

Images of successful 3D structures were captured immediately after their formation. The hydrogel formed in Run 7, produced a soft gel; the hydrogel in Run 8 produced a soft-to-firm gel; the hydrogel from Run 9 produced a firm gel; the hydrogel from Run 10 produced a soft gel (as shown in FIG. 6); the hydrogel from Run 11 produced a soft-to-firm gel; and the hydrogel from Run 12 produced a firm gel.

Rheology

Sections of the formed 3D cube were removed and rheology analysis was performed immediately after dispensing and at 30, 60, 90, 120, 180, 240 and 300 minutes.

FIG. 7 is a graph showing stiffness differences (kPa) between the three gel types (soft, soft-to-firm and firm), at the higher CaCl₂ concentration, and the stability over a short period of time.

Frequency sweeps were carried out from 0.1 Hz to 100 Hz, at constant oscillation amplitude and temperature, and with a working gap of 0.5 mm. From the graph of storage modulus against frequency produced, the stiffness of the sample was calculated. The geometry of the circular reader was PU20 (20 mm diameter).

FIG. 8 is a graph showing stiffness differences (kPa) between the three gel types (soft, soft-to-firm and firm) at the lower CaCl₂ concentration and the stability over a short period of time

Conclusion:

The described method used which simulates a two separate ‘ink’ cartridge set-up for printing was successful in producing a 3D cube structure. The gel material formed more or less instantaneously and rheology analysis was performed on sections of the 3D cube over a five-hour period. The 3D cubes for all concentrations of pre-gel and calcium solution retained their shape and rheology results showed that stiffness of the gel was satisfactory and stable over the period of time it was monitored.

At the lower concentrations of calcium chloride (i.e. 0.5 and 2 mM) there was no impact on the appearance of the pre-gel. However, as the concentration of calcium chloride was increased this resulted in the pre-gel becoming more opaque. An increase in pre-gel concentration lead to an increase in viscosity, due to the increased density of fibres present. The addition of the calcium chloride also resulted in an increase in the viscosity due to the cross-linking (or partial cross-linking) of the fibres. The viscosity is also affected by the printing process, for example by the printer head nozzle size, the distance to printing surface and the temperature. As a result the exact concentration is determined empirically for an individual printer.

Building on this, development was continued, moving to the exploration of layered 3D printing to create tubular and lattice constructs, allowing further optimisation of both gel crosslinking strength and printing parameter control.

### EXAMPLE 3

A third experimental procedure was then developed to use a 3D printer, and specifically one in which bio-ink is printed from a single nozzle from a single cartridge. Most 3D printers used in laboratories at the present time are fitted with a single print cartridge only; printers having two cartridges and thus able to print two materials simultaneously are more expensive—although this may change as this technique develops. Above experiments demonstrate that this new bio-ink can be used in printers having two cartridges. This set of experiments also demonstrates that the new bio-ink can be used with printing having a single cartridge only.

The printer used was a Cellink Ink-credible 3D printer.

This was used to investigate layered 3D printing to create tubular and lattice constructs, and gel formulations including additional elements.

Aim

To produce a material that could be printed from a single printer cartridge—through one nozzle—with no additional activator agent required at the point of printing.

This method was also used to investigated further combinations of bio-ink materials for their viscosity properties in comparison to two common printable materials, namely 4% alginate with 0.15% CaCl₂, and 40% Pluronic F127. The specific combinations of bio-ink materials were tested for their ability to print specific 3D shapes.

The additional materials included in the different combinations were the laminin sequence IKVAV lyophilised powder; 4% alginate with 0.15% CaCl₂; 40% Pluronic F127; DMEM media; PBS and Kolliphor P407.

Preparation of the Solutions

All samples were prepared in 1.5 mL Eppendorf tubes (with the exception of a scale-up sample which was prepared in a 15 mL falcon tube).

The viscosity when printed of various bio-inks was tested as set out in the table of FIG. 9. The constituents of the gets are set out in the Table and were all made as described below in relation to three examples.

Example 1-30 mM FFS with 0.25% (w/v) IKVAV addition+200 uL DMEM per 1 mL of pre-gel

Fmoc-FF and Fmoc-S were weighed so as to be present at a 1:1 molar ratio. 12.5 mg of IKVAV was added. The powders were dissolved and lyophilised.

157 mg of Biogelx lyophilised FFS/IKVAV powder (as described above) was weighed into a 15 mL Falcon tube and 5 mL dH₂O added. The tube was then vortexed and sonicated until no particulates remained. Following this, 1 mL of DMEM was added and the tube was vortexed again to mix. A final sonication was used to remove any pockets of air. The bio-ink was then be transferred to the printer cartridge.

Example 2-30 mM FFS in 1% Kolliphor P407+200 uL DMEM per 1 mL of pre-gel

A 20% stock solution of Kolliphor P407 was prepared by dissolving the powder in water at room temperature.

132 mg of Fmoc-FF and Fmoc-S (1:1) lyophilised powder was weighed into a 15 mL Falcon tube and 5 mL dH₂O added. The tube was then vortexed and sonicated until no particulates remain. Following this, 250 uL of the stock 20% Kolliphor P407 was added and the tube was vortexed to mix. 1 mL of DMEM was added and the tube was vortexed again. A final sonication was used to remove any pockets of air. The bio-ink could then be transferred to the printer cartridge.

Example 3-30 mM FFS+200 uL DMEM per 1 mL of pre-gel 132 mg of Fmoc-FF and Fmoc-S (1:1 lyophilised powder) was weighed into a 15 mL Falcon tube and 5 mL dH₂O added. The tube was then vortexed and sonicated until no particulates remain. Following this, 1 mL of DMEM was added and the tube was vortexed again to mix. A final sonication was used to remove any pockets of air. The bio-ink could then be transferred to the printer cartridge.

Results

As shown in FIG. 9, the prior art materials which have the highest viscosities immediately after printing are the currently two commonly used printing materials, namely 4% alginate with 0.15% CaCl₂ (which has a viscosity of 1583 Centipoise) and 40% Pluronic F127 (which has a viscosity of 4161 Centipoise). This allows them to hold their shape once printed.

For peptide-based gels of the invention, printable inks were obtained that were partially gelled and had initial viscosities of about 250-300 Centipoise or greater.

The resulting shortlisted library of combination materials were used to further explore their ability to create tubular and grid constructs using layered 3D printing by a 3D extrusion printer (Cellink—Inkredible). FIG. 10 shows a photograph of printing using Cellink Inkredible printer.

Printing fully gelled material resulted in the printer head acting as a filter, separating the water content of the gel from the fibres structure.

Printing the pre-gel on its own directly into a bath of DMEM media or printing pre-gel that had been reconstituted with diluted DMEM were both successful methods. For example a disturbed ring structure was obtained, and the material did gel. Thus, we used between 0 and 50% DMEM in subsequent experiments.

Following the further viscosity studies of combination materials it was decided that three specific combinations resulted in viscosities for further printing studies. These were 30 mM FFSIKVAV (namely Fmoc-FF, Fmoc-S and IKVAV) with 200 μL DMEM added per 1 mL of pre-gel (Run 13 and as described in Example 1); 30 mM FFS (namely Fmoc-FF with Fmoc-S) in 1% Kolliphor P407 and 200 μL DMEM added per 1 mL of pre-gel gel (Run 14 and as described in Example 2); and lastly 30 mM FFS and 200 μL DMEM added per 1 mL of pre-gel gel (Run 15 and as described in Example 3).

These materials were made by rehydrating FFS powder (1:1 Fmoc-FF and Fmoc-S) in dH₂O and then adding DMEM at 200 μL per mL of pre-gel. For Kolliphor P407 addition a stock 20% solution had been prepared previously and this was diluted (e.g. 1 in 20) with the pre-gel to achieve different working concentrations (i.e. 1 in 20 dilution would give a 1% solution). Then DMEM addition would follow at the same volume as previously stated for the other two samples. All samples were left overnight at room temperature for these investigations.

Printed rings, grids and cylinders achieved for all samples by successively building up layers of material. FIG. 11 shows a photograph of printed gel grid structure (5 layers) from Run 14; FIG. 12 shows a photograph of printed gel cylinder structure from Run 15; and FIG. 13 shows a photograph of printed gel grid structure (5 layers) from Run 15. The pressure required to print all materials was 4-fold less than the material reconstituted with 50% diluted media which is preferred for when printing cells as high pressure is likely to damage them. The constructs were reviewed following 1 hour incubation at room temperature post printing, demonstrating that the structures held their shape without additional cross-linker.

Peptide derivatives are stable as lyophilised powders and the provision of bio-inks in the form of powders, which can be made up by the addition of water, or culture media, represent a stable and convenient form for end users. As a result bio-inks that have the potential to be prepared for storage and transport in the form of a powder were investigated.

Bio-inks were created based on the materials discussed above, and two further combinations, namely one containing RGD (i.e. the tripeptide arginine-glycine-aspartic acid) and the other GrowDex® (i.e. hydrogel extracted from birch, and available from www.growdex.com). Bio-inks were created containing different combinations of components and were freeze dried into a lyophilised powder.

The following bio-inks were prepared:

A 30 mM FFS
52.8 mg of lyophilised FFS powder (FFS017MC) (ie a 1:1 lyophilised mixture of Fmoc-FF and Fmoc-S) was weighed into a tared 7 mL glass vial and 2 mL dH₂O was pipetted into the vial. Vortex and sonication was used to dissolve/rehydrate the powder. 400 μL DMEM was added by pipette to the vial and mixed by vortex, sonication was used to remove air bubbles.

B 30 mM FFSIKVAV (0.25% w/v)
63.2 mg of lyophilised FFS and IKVAV powder (FFSIKVAV001 MC) (i.e. a lyophilised mixture of 1:1 Fmoc-FF and Fmoc-S with 0.25% IKVAV) was weighed into a tared 7 mL glass vial and 2 mL dH₂O was pipetted into the vial. A magnetic bead was placed in the vial and the vial was placed on to a stirring platform which was used to dissolve/rehydrate the powder. 400 μL DMEM was added by pipette to the vial and it was mixed again using the magnetic bead, sonication was used to remove air bubbles.

C 30 mM FFSRGD (ratio 2:1:1)
86.9 mg of lyophilised FFS and RGD powder (FFSRGD001MC) was weighed into a tared 7 mL glass vial and 2 mL dH₂O was pipetted into the vial. Vortex and sonication was used to dissolve/rehydrate the powder. 400 μL DMEM was added by pipette to the vial and mixed by vortex, sonication was used to remove air bubbles.

D 30 mM FFS in 0.83% Kolliphor P407
52.8 mg of lyophilised FFS powder (FFSD017MC) was weighed into a tared 7 mL glass vial and 1.9 mL dH₂O was pipetted into the vial. Vortex and sonication was used to dissolve/rehydrate the powder. 100 μL of a 20% stock Kolliphor P407 solution was added by pipette to the vial and mixed by vortex, sonication was used to remove air bubbles. 400 μL DMEM was added by pipette to the vial and mixed by vortex, sonication was used to remove air bubbles.

E 30 mM FFS in GrowDex® (1/20 dilution)
52.8 mg of lyophilised FFS powder (FFSD017MC) was weighed into a tared 7 mL glass vial and 1.8 mL dH₂O was pipetted into the vial. Vortex and sonication was used to dissolve/rehydrate the powder. 200 μL of stock (as supplied 1.5% concentration) GrowDex® solution was added by pipette to the vial and mixed by vortex, sonication was used to remove air bubbles. 400 μL DMEM was added by pipette to the vial and mixed by vortex, sonication was used to remove air bubbles

All samples were stored in the fridge for 24 hours prior to viscosity measurement. Viscosity measurement was performed and the remainder of the samples were freeze dried. Following freeze drying all samples were used to prepare 1 mL of bio-ink in 1.5 mL Eppendorf tubes. These were left in the fridge overnight prior to repeating the viscosity measurements.

All bio-inks once prepared were observed to be pink in colour due to the phenol red in the DMEM media, indicating the pH of all materials to be similar and around neutral. On freeze drying all materials resulted in a fine powder that was observed to be white apart from the sample containing Kollifor P407 which had a pink tinge to it.

On rehydration of bio-inks with dH₂O all bio-inks returned to pink and had their previous appearance. The bio-ink containing laminin sequence IKVAV (namely sample B) was initially able to be vortexed however quickly became viscous to the point that vortexing resulted in bubbles which were only partially able to be removed through sonication. Following immediate preparation of the samples they were visually observed as not being as viscous as they were following the initial 24 hour incubation in the fridge so they were returned to the fridge for overnight incubation. The flowing day it was noted that the bio-ink containing Kollifor P407 (namely sample D) remained as a flowing solution. Once this sample had been removed from the fridge and had reached room temperature it had returned to the ‘set’ condition as prior to freeze drying.

As can be seen in the FIG. 14, the graph of the viscometry results, the viscometry of the bio-inks are comparable pre- and post-freeze drying. The only exception being the combination of Fmoc-FF/Fmoc-S and RGD which does shown a significant difference pre- and post-freeze drying, specifically that the gel is stiffer after freeze drying than prior to freeze drying. The increase in viscosity is also not a detrimental outcome as the viscometry value of ˜350 centipoise was still very much suitable for extrusion printing.

There was also a noticeable difference pre- and post-freeze drying with the combination of Fmoc-FF/Fmoc-S and IKVAV. In addition the bio-ink formed after freeze drying had a high variation in viscosity. It is likely that this is due to the difficulty when rehydrated the lyophilised powder and its high viscosity state.

The stability of the material over a longer time was then assessed. A 30 mM FFS bio-ink was prepared by the following method
52.8 mg of lyophilised FFS powder (FFS017MC) (i.e. a 1:1 mixture of Fmoc-FF and Fmoc-S) was weighed into a tared 7 mL glass vial and 2 mL dH₂O was pipetted into the vial. Vortex and sonication was used to dissolve/rehydrate the powder. 200 μL DMEM was added by pipette to the vial and mixed by vortex, sonication was used to remove air bubbles.

This material was stored at 4° C. for a three month period. The material was then removed from the fridge and allowed to come to room temperature before printing with a mechanical extrusion printer so as to form two rings one within the other. A small, 30 gauge, needle was used as the print head. Following printing the structure was immersed in DMEM to evaluate if it remained in the same construct or degraded.

As can be seen in FIG. 15 the material was printable and formed precise rings, one inside the other which required several layers to build up. Furthermore the construct remained stable following 24 hours immersion in DMEM.

Conclusion

A bio-ink has been developed that is printable from a single ‘ink’ cartridge. Increasing the viscosity to a threshold of 250-300 Centipoise for extrusion printing was achievable through addition of DMEM as well as other components such as laminin sequences (IKVAV), fibronectin and Kolliphor P407. In addition it has been possible to prepare bio-inks and freeze dry them for reconstitution by an end user, providing a stable and convenient form for storage and transport. Such bio-inks are stable under refrigeration conditions for periods of several months and are stable once printed.

A Cellink Inkredible 3D printer was used to print three materials successfully to form structures which required additional layers of bio-ink to be added and for them to fuse together to form a single construct.

### EXAMPLE 4

### Drug Release from Hydrogels

We tested a hydrogel of the invention for its ability to provide slow or delayed release of drug.

Aim

To evaluate the ability of Fmoc-FF/S hydrogel to provide sustained release of a model drug compound. The drugs studied were Fluvastatin, Pravastatin, Propranolol and Sotalol.

Experimental

The 4 model compounds used in this study are shown below.

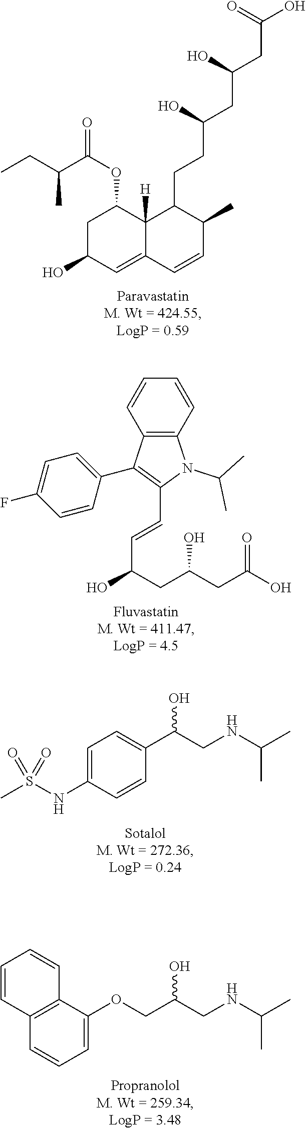 [SMILES: CC[C@H](C)C(=O)O[C@H]1C[C@H](O)C=C2C=C[C@H](C)C(CC[C@@H](O)C[C@@H](O)CC(=O)O)[C@H]21 ; CC(C)n1c(/C=C/[C@H](O)C[C@H](O)CC(=O)O)c(-c2ccc(F)cc2)c2ccccc21 ; CC(C)NCC(O)c1ccc(NS(C)(=O)=O)cc1 ; CC(C)NCC(O)COc1cccc2ccccc12]

Pre-Gel Preparation
A 2 mM solution of each of the above compounds in distilled water was prepared, and then each solution was added to a vial containing 17.2 mg of Biogelx gel powder.
The contents of each vial were alternately vortexed and sonicated for 30 secs-1 min to ensure the powder was completely dissolved and that a homogenous pre-gel solution was formed.
The pre-gel solutions with drug compounds incorporated were stored at 4° C. until required.
After overnight storage, the pre-gel solutions containing Fluvastatin, Pravastatin and Sotalol had become more viscous, but were still free flowing. The pre-gel solution containing Propranolol had become extremely viscous, and when the vial was inverted, the pre-gel was self-supporting (see results section for image).
A 2 mM control solution of each drug in PBS solution (pH 7.4) was also prepared.

Release Assay
The pre-gel solutions (containing drug compounds) were removed from cold storage, allowed to reach room temperature, and then vortexed and sonicated for 30 seconds to ensure a homogenous solution was achieved.
Release buffer (PBS) and 24 well plates were pre-warmed in an incubator at 37° C. in a humidified atmosphere of 5% CO₂.
500 μL of release buffer added to 6 wells of the plate and 1.5 mL of distilled water was added to the surrounding wells.
The plates were then equilibrated at 37° C. for 15 minutes.
For each plate, 50 μL of one of the pre-gel solutions containing a drug compound was added to a 24-well insert, and the insert placed in a well containing release buffer (performed in triplicate).
50 μL of each of the 2 mM control solutions was added to a 24-well insert and added to a well containing release buffer in the appropriate plate (performed in triplicate).
The plates were returned to the incubator for the required time.
At each specified time point (see results section), the inserts containing the gel samples and the controls were moved to a fresh 24-well plate prepared in the manner described above.
At each of the specified time points the release buffer remaining in the wells was transferred to HPLC vials and stored at 4° C. until HPLC analysis was performed.

HPLC Analysis

The samples of release buffer collected at each time point were analysed using

“Drug Release Assay” method at 220 nm.

An 1100 Series high-performance liquid chromatography pump (Agilent, USA) was used for the analysis which was performed using acetonitrile with 0.1% TFA and water with 0.1% TFA. The eluent solvent system had a linear gradient of 40% (v/v) acetonitrile in water for 3 mins, gradually rising to 80% (v/v) acetonitrile in water at 12 mins. This concentration was kept constant until 16 mins; after that the gradient was decreased, reaching 40% (v/v) acetonitrile in water at 19 mins. The data was then processed manually using the software ‘Agilent Chemstation.’

Results

Appearance of Pre-Gel Solutions

All solutions set to viscous gels. The propranolol gel seemed stiffest and retained its gel form even when inverted.

HPLC Analysis

The triplicate samples of release buffer were analysed by HPLC, and for the peaks corresponding to each model drug compound the peak area was measured. The average peak area was recorded, and using a standard curve prepared for each compound the concentration of each was determined.

Fluvastatin, Pravastatin and Sotalol

The data showed that for Fluvastatin, Pravastatin and Sotalol the release profile of the drugs from the hydrogel was slower than and the release time was extended compared to that from the control PBS solution. For propranolol both release time and profile altered significantly compared with the control and the propranolol-containing gel was identified for more detailed study.

Propranolol

The release profile for propranolol was examined in detail and found to be as follows in Table 5:

Retention time: 6.9-7.0 min

TABLE 5

| | |
|---|---|
| Average | Concentration |
| peak area | (ug/mL) |

| Timepoint | Control | Gel | Control | Gel |
|---|---|---|---|---|
| 30 min | 5761.4 | 262.5 | 17.8 | 0.03 |
| 3 h | 11536.5 | 2427.9 | 36.4 | 7.02 |
| 6 h | 271.1 | 1920.2 | 0.06 | 5.38 |
| 9 h | 11.9 | 1682.3 | 0 | 4.61 |
| 24 h | 0 | 2661.3 | 0 | 7.78 |
| 48 h | 0 | 2573.0 | 0 | 7.49 |
| 72 h | 0 | 1498.7 | 0 | 4.02 |
| 5 d | 0 | 1431.9 | 0 | 3.81 |
| 7 d | 0 | 886.5 | 0 | 2.05 |

Graphs of the release of Propranolol and cumulative release of Propranolol are shown in FIGS. 16 and 17.

For Propranolol a sustained release from the gels over the whole 7-day study was hence observed and, lasting in excess of 7 days, was notably extended in time compared with the control.

Two further runs using propranolol were tested, differing only in that the concentrations of the peptides were 10 mM and 40 mM (rather than 20 mM in the example above). In both cases, significant drug concentration continued to be released after 72 hours and more, compared with the control in which drug release was below measurable limits by the 9 hour point.

Conclusion

The Fmoc-FF/S hydrogels were prepared containing a range of different drugs, giving clear, viscous and stable gels. Release of drug from these was extended compared with controls and significantly so in the case of propranolol.

Thus both examples demonstrated that extrusion of hydrogel precursors with cations can be used to produced 3D printed hydrogel structures and structures effective for slow/delayed release of drugs.

### EXAMPLE 5

### Further Drug Release from Hydrogels Study

Following the success of the first study set out above a further study was conducted using compounds.

Aim:

To evaluate the ability of Fmoc-FF/S hydrogels to provide sustained release of model compounds possessing specific structural features to gain a better understanding of the observed sustained release of previously screened drug Propranolol.

Experimental:

The 2 model compounds (Betaxolol and Quinidine) used in this study are shown below along with the previously studied Propranolol.

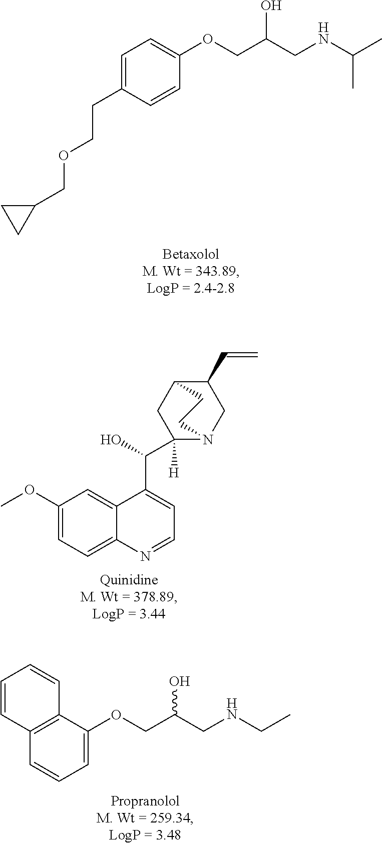

Pre-Gel Preparation

Betaxolol Pre-Gel
A 2 mM solution of each of the above compounds in distilled water was prepared, and then each solution was added to a vial containing 17.2 mg of Biogelx powder, namely lyophilized Fmoc-FF/Fmoc-S.
The contents of each vial were alternately vortexed and sonicated for 30 secs-1 min to ensure the powder was completely dissolved and that a homogenous pre-gel solution was formed.
The pre-gel solutions with drug compounds incorporated were stored at 4° C. until required.
After overnight storage, the pre-gel solution containing Betaxolol had become significantly more viscous, and when the vial was inverted, the pre-gel was self-supporting (see results section for image).
A 2 mM control solution of Betaxolol in PBS solution was also prepared.

Quinidine Pre-Gel
A 2 mM solution of Quinidine in distilled water was prepared, and the solution was added to a vial containing 17.2 mg of Biogelx gel powder. A white suspension was formed.
The contents of each vial were alternately vortexed and sonicated for several minutes, however the solid would not dissolve.
The pH of the solution was adjusted using 0.5M NaOH. At approx. pH 10 a cloudy slightly viscous solution was formed.
The pre-gel solution with drug compound incorporated was stored at 4° C. until required.
After overnight storage, the pre-gel solution containing quinidine did not look significantly different, although the pH had fallen to approx. 9.
A 2 mM control solution of Quinidine in PBS solution was also prepared.

Release Assay
The pre-gel solutions (containing drug compounds) were removed from cold storage, allowed to reach room temperature, and the vortexed and sonicated for 30 seconds to ensure a homogenous solution was achieved.
Release buffer (PBS) and 24 well plates were pre-warmed in an incubator at 37° C. in a humidified atmosphere of 5% CO₂.
500 uL of release buffer added to 6 wells of the plate and 1.5 mL of distilled water was added to the surrounding wells.
The plates were then equilibrated at 37° C. for 15 minutes.
For each plate, 50 uL of one of the pre-gel solutions containing a drug compound was added to a 24-well insert, and the insert placed in a well containing release buffer (performed in triplicate).
50 uL of each of the 2 mM control solutions was added to a 24-well insert and added to a well containing release buffer in the appropriate plate (performed in triplicate).
The plates were returned to the incubator for the required time.
At each specified time point (see results section), the inserts containing the gel samples and the controls were moved to a fresh 24-well plate prepared in the manner described above.

At each of the specified time points the release buffer remaining in the wells was transferred to HPLC vials and stored at 4° C. until HPLC analysis was performed.

HPLC Analysis

The samples of release buffer collected at each time point were analysis using “Drug Release Assay” method at 220 nm.

An 1100 Series high-performance liquid chromatography pump (Agilent, USA) was used for the analysis which was performed using acetonitrile with 0.1% TFA and water with 0.1% TFA. The eluent solvent system had a linear gradient of 40% (v/v) acetonitrile in water for 3 mins, gradually rising to 80% (v/v) acetonitrile in water at 12 mins. This concentration was kept constant until 16 mins when the gradient was decreased to 40% (v/v) acetonitrile in water at 19 mins. The data was then processed manually using the software ‘Agilent Chemstation.’

Results:

Appearance of Pre-Gel Solutions

The Betaxolol containing solution set to a self-supporting gel, while Quinidine contain solution formed a viscous solution.

HPLC Analysis

The triplicate samples of release buffer were analysed by HPLC, and for the peaks corresponding to each model drug compound the peak area was measured. The average peak area was recorded, and using standard curve prepared for each compound the concentration of each was determined. Table and graphs of this data for each model compound can be seen below.

Betaxolol

The release time is set out in the Table 6 below, which as can be seen demonstrates an overall retention time of 7.4 min.

TABLE 6

| | |
|---|---|
| Average | Concentration |
| peak area | (ug/mL) |

| Timepoint | Control | Gel | Control | Gel |
|---|---|---|---|---|
| 30 min | 1682.37 | 109.83 | 10.34 | 0.67 |
| 3 h | 3685.33 | 1477.90 | 22.71 | 9.08 |
| 6 h | 147.87 | 1037.33 | 0.90 | 6.37 |
| 9 h | 12.10 | 486.83 | 0.07 | 2.98 |
| 24 h | 0 | 839.17 | 0 | 5.15 |
| 48 h | 0 | 244.40 | 0 | 1.49 |
| 72 h | 0 | 44.37 | 0 | 0.27 |
| 5 d | 0 | 17.43 | 0 | 0.11 |
| 7 d | 0 | 0 | 0 | 0 |

Graphs of the release of Betaxolol and cumulative release of Betaxolol are shown in FIGS. 18 and 19.

Quinidine

The release time is set out in the Table 7 below, which as can be seen demonstrates an overall retention time of 8.5 min.

TABLE 7

| | |
|---|---|
| Average | Concentration |
| peak area | (ug/mL) |

| Timepoint | Control | Gel | Control | Gel |
|---|---|---|---|---|
| 30 min | 3325.47 | 340.80 | 7.26 | 1.82 |
| 3 h | 6122.67 | 581.77 | 32.25 | 3.10 |
| 6 h | 86.37 | 622.87 | 0.47 | 3.32 |
| 9 h | 7.77 | 421.20 | 0.04 | 2.25 |
| 24 h | 0 | 960.23 | 0 | 5.11 |
| 48 h | 0 | 1170.43 | 0 | 6.22 |
| 72 h | 0 | 778.67 | 0 | 4.15 |
| 5 d | 0 | 406.67 | 0 | 2.17 |
| 7 d | 0 | 558.00 | 0 | 2.98 |

Graphs of the release of Quinidine and cumulative release of Quinidine are shown in FIGS. 20 and 21.

*It was noted that the results for the Quinidine Control corresponded to only 85% release.

Discussion

Comparing the data above, to the previously tested Propranolol (data shown below in FIGS. 16 and 17), it can be seen that both Betaxolol (FIGS. 18 and 19) and Quinidine (FIGS. 20 and 21) have a similar release profile to Propranolol.

Betaxolol was released significantly more slowly from the 10 mM Fmoc-FF/S gel than from the control insert (5 d vs. 9 h) however this release was over a shorter time than Propranolol, which showed a sustained release over the full 7-day study. Betaxolol, contains a similar amine chain to Propranolol, but only contains a single aromatic ring. The release results of Betaxolol indicate that although a conjugated aromatic system isn't necessary for sustained release of a molecule, the extent of aromaticity does influence the release, as Betaxolol with a single aromatic ring is release more quickly than Propranolol with a naphthalene system (2 rings).

Quinidine was also released significantly more slowly from the 10 mM Fmoc-FF/S gel than from the control, with sustained release being observed over the whole 7-day study, compared to 9 h for the control. HPLC analysis showed that 4.5% of the drug was still present in the remaining gel residue at the end of the experiment. Like Propranolol, Quinidine is a weakly basic compound with a conjugated aromatic system, but with a different chemotype. The release profile of this compound suggests that it is these general features which result in sustained release from the Fmoc-FF/S system, as opposed to specifically Propranolol-like structures.

Conclusion

It has been demonstrated that Fmoc-FF/S hydrogels provided steady, slow release rates of the additional model compounds, Betaxolol and Quinidine over simple diffusion. Both show a similar release profile to Propranolol, with sustained release of Quinidine occurring over the entire 7-day study and Betaxolol being released at a slightly faster rate (over 5 days). These results indicate that the extent of aromaticity within the encapsulated molecule may influence the release rate of the molecule, particularly when comparing the release of Propranolol and Betaxolol which possess similar chemotypes.

The invention hence provides 3D printing of peptide-containing hydrogels, and uses of those gels e.g. for cell growth and drug delivery.

1. A method of printing, comprising:
a. preparing ink comprising a mixture of hydrogel precursor and activator in non-gelled form,
b. printing the ink onto a substrate, and
c. allowing the ink to gel,
wherein the hydrogel precursor comprises a plurality of peptide derivatives and the activator comprises a cross-linking agent

2. A method according to claim 1, wherein preparing the ink comprises combining the hydrogel precursor with the activator so that it begins to gel, and printing the gelling ink before it has gelled.

3. A method according to claim 1, wherein preparing the ink comprises combining the hydrogel precursor with the activator so that it partially gels, and allowing the ink to gel comprises combining the partially gelled ink with further activator so that a gel is formed.

4. A method according to any preceding claim, wherein the ink has a viscosity of at least 150 cP.

5. A method according to any preceding claim, wherein the ink has a viscosity of at least 250 cP.

6. A method according to any preceding claim, comprising co-printing or coextruding hydrogel precursor and activator from separate reservoirs.

7. A method of printing, comprising
a. preparing ink comprising a hydrogel precursor in non-gelled form,
b. printing the ink so as to contact it with activator, and
c. allowing the ink to gel,
wherein the hydrogel precursor comprises a plurality of peptide derivatives and the activator comprises a cross-linking agent.

8. A method according to claim 7, comprising printing into a solution containing the activator.

9. A method according to claim 7, comprising co-printing or coextruding hydrogel precursor and activator from separate reservoirs.

10. A method according to any previous claim, wherein the peptide derivatives comprise at least 2 peptides linked to a third component which is an aromatic peptide or is an aromatic stacking ligand.

11. A method according to any previous claim, wherein the activator comprises a cross-linking agent, e.g. a cation.

12. A method according to any previous claim, wherein the peptide derivatives are of formula I
ASL-GA-GA-X₍ₙ₎ (I)
wherein
ASL is an aromatic stacking ligand comprising an aromatic group, each GA is independently an amino acid or a derivative thereof, X is an amino acid or a derivative thereof, n is an integer from 0 to 3, and wherein the peptide derivatives form a gel in the presence of crosslinking cations.

13. A method according to claim 12, wherein ASL is Fmoc, CBz, an aromatic amino acid, or a derivative thereof.

14. A method according to claim 12 or 13, wherein the peptides derivatives are of formula Ia
ASL-GA1-GA2-X₍ₙ₎ (Ia)
wherein
GA1 is selected from phenylalanine (F), tyrosine (Y) and tryptophan (W) and derivatives thereof,
Each GA2 and X is independently selected from
(1) neutral amino acids alanine (A), leucine (L), asparagine (N), methionine (M), cysteine (C), glutamine (Q), proline (P), glycine (G), serine (S), isoleucine (I), threonine (T), tyrosine (Y), tryptophan (W) and valine (V), positively charged amino acids arginine (R), histidine (H) and lysine (K), and negatively charged amino acids aspartic acid (D) and glutamic acid (E), and derivatives thereof, and (2) phenylalanine (F), tyrosine (Y) and tryptophan (W) and derivatives thereof, and
n is an integer from 0 to 3.

15. A method according to claim 14, wherein the peptides comprise a mixture of peptides, some of which comprise a GA2 from list (1) and some of which comprise a GA2 from list (2).

16. A method according to any previous claim wherein the ink further comprises cells.

17. A method according to any previous claim, wherein the ink further comprises an active agent.

18. A method according to any previous claim, comprising printing a structure containing an active agent, for sustained delivery of the active agent.

19. A method according to any previous claim, comprising printing a cell support structure containing cells.

20. A method according to any previous claim, comprising printing a sheet of hydrogel precursor.

21. A method according to any previous claim, comprising
printing a first layer of ink,
allowing the ink to partially gel, and
printing a second layer of ink in contact with the first layer.

22. A method according to any previous claim, wherein the gelled hydrogel has a stiffness of 5 kPa or greater.

23. A method according to any previous claim, wherein the gelled hydrogel has a stiffness of 10 kPa or greater.

24. Printer ink, comprising hydrogel precursor, wherein the hydrogel precursor comprises a plurality of peptide derivatives and forms a gel in contact with a cross-linking agent.

25. Printer ink according to claim 24, further comprising cells.

26. Printer ink according to claim 24 or 25, further comprising an active agent.

27. Printer ink according to any of claims 24 to 26, further comprising a cross-linking agent.

28. Printer ink according to any of claims 24 to 27, wherein the peptide derivatives comprise at least 2 peptides linked to a third component which is an aromatic peptide or is an aromatic stacking ligand.

29. A composition comprising hydrogel precursor and active agent for use in sustained release delivery of the active agent, wherein the hydrogel precursor comprises a plurality of peptide derivatives and forms a gel in contact with a cross-linking agent.

30. A composition for use according to claim 29, wherein the peptide derivatives each independently comprise at least 2 peptides linked to a third component which is an aromatic peptide or is an aromatic stacking ligand.

31. A composition for use according to claim 29 or 30, having a stiffness of 5 kPa or greater.

32. A composition for use according to claim 29 or 30, having a stiffness of 10 kPa or greater.

33. A composition for use according to any of claims 29 to 32, wherein the sustained release of the active agent is over a period of 2 days or more.

34. A composition for use according to any of claims 29 to 33, wherein the sustained release of the active agent is over a period of 5 days or more.

35. A composition for use according to any of claims 29 to 34 by injection of hydrogel precursor into a patient wherein the hydrogel gels in situ.

35. A composition for use according to any of claims 29 to 34 by surgical insertion of gelled hydrogel into a patient.

36. A method of sustained delivery of an active agent to a patient, comprising administering to the patient a hydrogel or precursor containing the active agent, wherein the hydrogel or precursor comprises a plurality of peptide derivatives and forms a gel in contact with a cross-linking agent.

37. A method according to claim 36, wherein the peptide derivatives each independently comprise at least 2 peptides linked to a third component which is an aromatic peptide or is an aromatic stacking ligand.

38. A method according to claim 36 or 37, wherein the hydrogel has a stiffness of 5 kPa or greater.

39. A method according to claim 38, wherein the hydrogel a stiffness of 10 kPa or greater.

40. A method according to any of claims 36 to 39, wherein the sustained release of the active agent is over a period of 2 days or more.

41. A method according to any of claims 36 to 40, wherein the sustained release of the active agent is over a period of 5 days or more.

42. A method according to any of claims 36 to 41, wherein administering is by injection of hydrogel precursor into a patient wherein the hydrogel gels in situ.

43. A method according to any of claims 36 to 41, wherein administering is by surgical insertion of gelled hydrogel into a patient.

44. A composition for use according to any of claims 29 to 35 or a method according to any of claims 36 to 40, wherein the peptides are of formula I
ASL-GA-GA-X₍ₙ₎ (I)
wherein
ASL is an aromatic stacking ligand comprising an aromatic group, each GA is independently an amino acid or a derivative thereof, X is an amino acid or a derivative thereof, n is an integer from 0 to 3, and wherein the peptide derivatives form a gel in the presence of crosslinking cations.

45. A composition for use according to claim 44 or a method according to claim 44, wherein ASL is Fmoc, CBz, an aromatic amino acid, or a derivative thereof.

46. A composition for use according to claim 44 or 45 or a method according to claim 44 or 45, wherein the peptides are of formula Ia
ASL-GA1-GA2-X₍ₙ₎ (Ia)
wherein
GA1 is selected from phenylalanine (F), tyrosine (Y) and tryptophan (W) and derivatives thereof,
Each GA2 and X is independently selected from
(1) neutral amino acids alanine (A), leucine (L), asparagine (N), methionine (M), cysteine (C), glutamine (Q), proline (P), glycine (G), serine (S), isoleucine (I), threonine (T), tyrosine (Y), tryptophan (W) and valine (V), positively charged amino acids arginine (R), histidine (H) and lysine (K), and negatively charged amino acids aspartic acid (D) and glutamic acid (E), and derivatives thereof, and (2) phenylalanine (F), tyrosine (Y) and tryptophan (W) and derivatives thereof, and
n is an integer from 0 to 3.

47. A composition for use according to claim 46 or a method according to claim 46, wherein the peptides comprise a mixture of peptides, some of which comprise a GA2 from list (1) and some of which comprise a GA2 from list (2).

48. Apparatus for creating a cell growth support structure, comprising a container of printer ink according to any of claims 24 to 28, linked to an extruder capable of extruding the ink.

49. Apparatus according to claim 48, further comprising a container of activator, linked to an extruder capable of extruding the agent so that is contacts extruded precursor.
